(19) United States
(12) Patent Application Publication
(10) Pub. No.: US 2003/0166561 A1
(43) Pub. Date: Sep. 4, 2003

(54) Peptide

(76) Inventors: Garth J. S. Cooper, Auckland (NZ); Christina M. Buchanan, Auckland (NZ)

Correspondence Address: Randolph Ted Apple, Morrison & Foerster LLP, 755 Page Mill Road, Palo Alto, CA, 94304-1018, US

(21) Appl. No.: 10/374,624

(22) Filed: Feb. 24, 2003

Related U.S. Application Data
(63) Continuation of application No. 09/745,078, filed on Dec. 20, 2000, now abandoned.

(30) Foreign Application Priority Data
Jun. 18, 1999 (NZ) NZ33659
Jun. 19, 2000 (WO) PCT/NZ00/00102

(51) Int. Cl. A61K 38/17; C07K 14/47

(52) U.S. Cl. 514/12; 530/324

(57) ABSTRACT

The invention relates to a bioactive mammalian peptide. In particular, it relates to a peptide secreted by the pancreatic islet β-cell that stimulates insulin secretion termed preptin.

Preptin analogs, pharmaceutical compositions which contain preptin or its analogs and their use as medicaments are inter alia also provided.

### CROSS REFERENCE TO RELATED APPLICATIONS

This application claims the priority benefit of New Zealand application number NZ336359, filed on Jun. 18, 1999 and PCT NZ00/00102, filed on Jun. 19, 2000, both of which are incorporated by reference in their entirety.

### FIELD OF THE INVENTION

This invention relates to a bioactive peptide. In particular, it relates to a peptide secreted by the pancreatic islet β-cell that stimulates insulin secretion.

### BACKGROUND

Pancreatic islet β-cells play a major regulatory role in physiology, mainly through their secretion of insulin, a peptide hormone which exerts profound effects on intermediary metabolism (Draznin et al (1994)). A second β-cell hormone, amylin, may also contribute to β-cell regulatory function through its actions on insulin secretion and tissue insulin sensitivity (Cooper, G (1994); Hettiarachchi et al (1997)).

In islet β-cells, hormones are packaged in secretory granules, which undergo regulated release in response to signals such as fuels (e.g., glucose, amino acids) or neurohormonal stimuli. These granules contain dense cores rich in insulin and Zn, while smaller amounts of insulin C-peptide, amylin, proinsulin, chromogranin-derived peptides, proteases and other proteins are found in the granule matrix (Hutton, J (1989)).

What the applicants have now determined is that pancreatic islet β-cells secrete another regulatory peptide. The applicants have further determined that this peptide enhances glucose-mediated insulin secretion.

It is generally towards this peptide, which the applicants have termed preptin, that the present invention is directed in its various aspects.

### SUMMARY OF THE INVENTION

Preptin is a previously unknown, pancreatic islet β-cell hormone. It is produced from the E-peptide of pro-IGF-II, is present in islet β-cell granules in significant amounts is co-secreted with insulin in a regulated manner, and enhances glucose-stimulated insulin secretion.

Accordingly, in a first aspect the present invention provides the peptide preptin or an analog thereof.

By “preptin”, the applicants mean a peptide of 34 amino acids, the sequence of which is as follows:

(SEQ ID No:1)

Asp Val Ser Thr R₁ R₂ R₃ Val Leu Pro Asp R₄ Phe
Pro Arg Tyr Pro Val Gly Lys Phe Phe R₅ R₆ Asp Thr
Trp R₇ Gln Ser R₈ R₉ Arg Leu

wherein:

R₁ is Ser or Pro;
R₂ is Gln or Pro;
R₃ is Ala or Thr;
R₄ is Asp or Asn;
R₅ is Gln or Lys;
R₆ is Tyr or Phe;
R₇ is Arg or Lys;
R₈ is Ala or Thr; and
R₉ is Gly or Gln.

or an analog thereof.

In one embodiment, the invention provides human preptin having the amino acid sequence:

(SEQ ID No:2)

Asp Val Ser Thr Pro Pro Thr Val Leu Pro Asp Asn
Phe Pro Arg Tyr Pro Val Gly Lys Phe Phe Gln Tyr
Asp Thr Trp Lys Gln Ser Thr Gln Arg Leu.

In another embodiment, the invention provides rat preptin having the amino acid sequence:

(SEQ ID No:3)

Asp Val Ser Thr Ser Gln Ala Val Leu Pro Asp Asp
Phe Pro Arg Tyr Pro Val Gly Lys Phe Phe Lys Phe
Asp Thr Trp Arg Gln Ser Ala Gly Arg Leu.

In yet another embodiment, the invention provides mouse preptin having the amino acid sequence:

(SEQ ID No:4)

Asp Val Ser Thr Ser Gln Ala Val Leu Pro Asp Asp
Phe Pro Arg Tyr Pro Val Gly Lys Phe Phe Gln Tyr
Asp Thr Trp Arg Gln Ser Ala Gly Arg Leu.

The amino acid sequence corresponds to Asp₆₉-Leu₁₀₂ of the proIGF-II E-peptide in each mammal.

In still a further aspect, the present invention provides a polynucleotide which encodes preptin or an analog thereof.

In another aspect, the invention provides a polynucleotide which codes for a bioactive fragment of preptin or preptin analog thereof.

In another aspect, the invention provides a vector which comprises a polynucleotide which encodes preptin or an analog thereof and which is capable of expressing preptin or said analog.

In another aspect, the invention provides a vector which comprises a polynucleotide which codes for a bioactive fragment of preptin or preptin analog thereof.

In yet another aspect, the invention provides for a host cell comprising a vector which comprises a polynucleotide which codes for preptin, a bioactive fragment of preptin, preptin analog, or a bioactive fragment of preptin analog thereof.

Preptin salts, which are preferably physiologically acceptable, are also provided.

In a further aspect, the invention further provides a pharmaceutical composition which comprises preptin or an analog thereof, or preptin salts.

In still a further aspect, the invention provides a method of stimulating insulin secretion for a therapeutic or prophylactic purpose which comprises the step of administering to a patient in need of such therapy or prophylaxis an effective amount of preptin or an analog thereof.

In yet a further aspect, the invention provides the use of preptin or an analog thereof or a salt thereof in the preparation of a medicament, particularly for stimulating insulin secretion.

In still a further aspect, the invention provides a method of modulating glucose mediated insulin secretion which comprises the step of administering to a patient an effective amount of preptin, a preptin analog, a preptin agonist or a preptin antagonist.

In another embodiment, the invention provides for methods for generating antibodies reactive against preptin comprising administering preptin, preptin analog, or an analog thereof as an immunogen.

In yet further embodiments, the invention provides antibodies which bind preptin or its analogs, assays which employ such antibodies and assay kits which contain such antibodies.

The above summary is not exhaustive. Other aspects of the invention will be apparent from the following description, and from the appended claims.

### BRIEF DESCRIPTION OF THE FIGURES

FIG. 1 shows purification and characterization of preptin. a) assays for marker proteins indicating the localization of organelles from βTC6-F7 cells within the continuous OptiPrep gradient; granule core (insulin), granule matrix (amylin), lysosomes (aryl sulphatase), mitochondria (citrate synthase). b) Granule proteins purified by RP-HPLC. The indicated peak (hatched) was collected and further purified. c) Purity and mass (M+H⁺) of the major peptide from the hatched peak confirmed by MALDI-TOF MS. d) RP-HPLC profile from the Lys-C digest of the peptide purified from the hatched peak. 1: NH₂-terminal fragment; 2: COOH-terminal fragment; 3: undigested peptide. e) Structure of mouse preptin as determined by sequencing of Lys-C-derived peptides from (d): NH₂-terminal fragment: normal font; COOH-terminal fragment: italicized-bold, and its localization in a segment of murine proIGF-II E-peptide shown. Domains of proIGF-II (B, C, A, D, E) are indicated. Recognized cleavage site at Arg₆₈ is indicated in bold, while putative dibasic motifs are shown as discontinuous lines.

FIG. 2 shows cellular preptin secretion. a) Preptin RIA standard curve. b) RIA characterization of preptin-like immunoreactive material. (PLIM) in RP-HPLC fractions of 24-h βTC6-F7 conditioned medium and intra-granular fractions from FIG. 1b. c) MALDI-TOF MS of the major PLIM containing fraction secreted from βTC6-F7 cells. Peak corresponds to murine preptin (M+H⁺) with 0.07% error.

FIG. 3 shows the effects of preptin on insulin secretion. a) Preptin-mediated insulin secretion from βTC6-F7 cells. Graph illustrates increments in insulin concentration above basal (0 added preptin). b) preptin-mediated insulin secretion from isolated perfused rat pancreas. Points are mean±SEM (duplicate analyses; n=4 pancreases for each curve). Area under curve (second phase of insulin secretion P=0.03 unpaired 2-tailed t-test).

FIG. 4 shows the immunohistochemistry of murine pancreas. Pancreas harvested from adult FVB/n mice was sectioned and stained with hematoxylin and polyclonal rabbit antisera using immunoperoxidase-conjugated goat-anti-rabbit second antibody. Panels are: a, anti-insulin antiserum (1:40); b, anti-preptin antiserum. (1:40); c,d, anti-preptin antiserum (1:40) pre-incubated for 30 min with synthetic rat preptin at c, 1 mg.ml⁻¹, d, 5 mg.ml⁻¹. Bar=100 μm.

FIG. 5 shows the RIA characterization of preptin-like immunoreactive material (PLIM) in RP-HPLC fractions from rat islets or βTC6-F7 granule fractions (standard; FIG. 1b).

FIG. 6 shows preptin and insulin co-secretion from βTC6-F7 cells and isolated rat islets. a,b Glucose-mediated co-secretion of preptin with insulin from a, βTC6-F7 cells and b, isolated rat islets.

FIG. 7 shows the effects of preptin on insulin secretion. a, b, Purity and mass of purified a, rabbit anti-rat preptin γ-globulin and b, non-immune rabbit γ-globulin. 1: light chain IgG, M+H⁺; 2: whole IgG, M+4H⁺; 3: heavy chain IgG, M+H⁺; 4: whole IgG, M+2H⁺; whole IgG, M+H⁺. c, 1-min preptin-binding capacity of perfused anti-preptin γ-globulin at 35 μg.ml⁻¹, 37° C., pH 7.4 to simulate contact time, dilution, temperature and pH of the antibody perfusion experiments. d, Effect of infusion of anti-preptin γ-globulin or control (non-immune rabbit γ-globulin) on insulin secretion from glucose-stimulated (20 mM; square wave) isolated perfused rat pancreases. Each point is mean±SEM (duplicate analyses; n=5 pancreases per curve). AUC (second phase of insulin secretion; P=0.03, unpaired 1-tailed t-test).

### DESCRIPTION OF THE INVENTION

The present invention is directed to a novel peptide which has been found in pancreatic islet β-cell granules. This peptide, preptin, has been determined to stimulate glucose-evoked insulin secretion. Preptin acts to recruit, prime and co-ordinate the glucose-responsive activity of β-cells in a local manner, amplifying the glucose-evoked signal to the β-cell organ. This action would be similar to the feed-forward mechanism effected in platelets by the thrombin-elicited release of thromboxane A₂ (Barritt (1992)).

The existence of a previously unsuspected mechanism, through which a new islet β-cell hormone amplifies glucose-mediated insulin secretion, suggests that preptin biology will be important in type 2 diabetes mellitus, which is characterized by a complex impairment of insulin secretion (De Fronzo et al (1992)) together with defects in insulin action (Cotran et al. “Pathologic Basis of Disease” 4th ed. 1989). A defect in preptin synthesis, secretion, or action could contribute to the defective glucose-mediated insulin secretion in this condition and preptin administration may be advantageous for the treatment of type 2 diabetes mellitus or other disorders associated with diminished β-cell insulin secretion. It is noted that, in humans, the variable number of tandem repeat (VNTR) polymorphism upstream of the adjacent insulin (INS) and IGF-II germs regulates expression of both genes, and is associated with an increased tendency to both type 2 diabetes mellitus and polycystic ovary syndrome.

General Techniques

The practice of the present invention will employ, unless otherwise indicated, conventional techniques of molecular biology (including recombinant techniques), microbiology, cell biology, biochemistry and immunology, which are within the skill of the art. Such techniques are-explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C.C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press NY, 1994); Antibodies: A Laboratory Manual (Harlow et al., eds., 1987); and Methods of Immunological Analysis (R. Masseyeff, W. H. Albert, and N. A. Staines, eds., Weinheim: VCH Verlags gesellschaft mbH, 1993).

Definitions

An “antibody” (interchangeably used in plural form) is an immunoglobulin molecule capable of specific binding to a target, such as a carbohydrate, polynucleotide or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity.

An “effective amount” is an amount sufficient to effect beneficial or desired results including clinical results. An effective amount can be administered in one or more administrations by various routes of administration.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

As used herein, “treatment” is an approach for obtaining beneficial or desired results including and preferably clinical results.

The terms “polypeptide” and “peptide” and the like are used interchangeably herein to refer to any polymer of amino acid residues of any length. The polymer can be linear or non-linear (e.g., branched), it can comprise modified amino acids or amino acid analogs, and it can be interrupted by chemical moieties other than amino acids. The terms also encompass an amino acid polymer that has been modified naturally or by intervention; for example, by disulfide bond formation, glycosylation, lipidation, acetylation, phosphorylation, or any other manipulation or modification, such as conjugation with a labeling or bioactive component.

The terms “polynucleotide” and “nucleic acid” used interchangeably herein, refer to a polymeric form of nucleotides of any length, either ribonucleotides, or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. It is understood that the double stranded polynucleotide sequences described herein also include the modifications described herein. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH2) or a mixed phosphoramidate-phosphodiester oligomer. A phosphorothioate linkage can be used in place of a phosphodiester linkage. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers.

A “vector” is a self-replicating nucleic acid molecule that transfers an inserted: nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions.

An “individual” is a vertebrate, preferably a mammal, more preferably a human. Mammals include, but are not limited to, farm animals, sport animals, pets, primates, mice and rats.

As used herein, “substantially pure”, or “essentially pure” preptin or preptin analog refers to a composition which is comprised of at least about 50% preptin, preferably at least about 85% preptin, preferably at least about 85% preptin, preferably at least about 85% preptin, preferably at least about 90%, more preferably at least about 95%. As used herein, “pure” preptin refers to a composition which is comprised of at least 95% preptin and more preferably at least about 99% preptin.

As used herein, the singular form “a”, “an”, and “the” includes plural references unless indicated otherwise. For example, “an” antibody includes one or more antibodies.

“Comprising” means including.

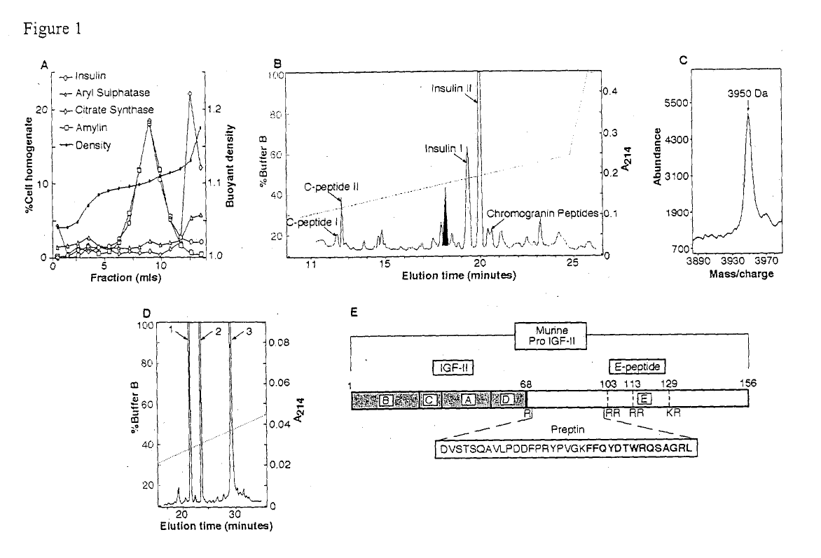

Preptin was identified using a single-step density-gradient centrifugal method to purify secretory granules from cultured murine βTC6-F7 cells with purity being confirmed by marker-protein analysis (FIG. 1a). Insulin was used to track purification of granule-cores, whereas amylin, which is present in the granule-matrix (Johnson, K (1988)), was measured to verify granule-membrane integrity (FIG. 1a). Soluble granule components were then separated using reversed-phase HPLC (A₂₁₄; FIG. 1b). Peptide-identity was determined by mass spectrometry and NH₂-terminal amino-acid sequencing. Major peaks contained murine insulins-I and -II and C-peptides-I and -II (FIG. 1a). No non-β-cell peptides were detected and the molar ratio of amylin:insulin (1:23) and mouse insulin I:mouse insulin II (1:3) were equivalent to those of physiological β-cells (Cooper (1994); Linde 1989)).

A major peak eluting immediately prior to insulin-I was found to contain a previously unknown peptide (FIG. 1b). This was purified to homogeneity and had a molecular mass of 3950 Da (FIG. 1c). The molecule was digested with a lysine-specific protease, and the resulting peptides separated by RP-HPLC (FIG. 1d) prior to complete NH₂-terminal protein sequencing. The complete sequence confirmed that the molecule contained 34-amino acids, which corresponded to Asp₆₉-Leu₁₀₂ of 10 murine proIGF-II E-peptide (FIG. 1e). This peptide is mouse preptin.

Preptin is flanked NH₂-terminally by a recognized Arg cleavage-site, and COOH-terminally by a putative dibasic (Arg-Arg) cleavage motif (Bell et al. (1985)) (FIG. 1e). These residues are highly conserved between species, and are likely to serve as post-translational processing signals.

While others have shown the existence of different proIGF-II E-peptide-derived peptides in cell culture medium and various mammalian biological fluids (Hylka et al. (1985), Daughaday et al (1992), and Liu et al. (1993)), none have identified one that is equivalent to preptin.

The amino acid sequence of mouse preptin is as follows:

(SEQ ID No:4)

Asp Val Ser Thr Ser Gln Ala Val Leu Pro Asp Asp
Phe Pro Arg Tyr Pro Val Gly 25 Lys Phe Phe Gln Tyr
Asp Thr Trp Arg Gln Ser Ala Gly Arg Leu

The equivalent amino acid sequences for human and rat preptin are, respectively:

(SEQ ID No:2)

Asp Val Ser Thr Pro Pro Thr Val Leu Pro Asp Asn
Phe Pro Arg Tyr Pro Val Gly Lys Phe Phe Gln Tyr
Asp Thr Trp Lys Gln Ser Thr Gln Arg Leu; and

(SEQ ID No:3)

Asp Val Ser Thr Ser Gln Ala Val Leu Pro Asp Asp
Phe Pro Arg Tyr Pro Val Gly Lys Phe Phe Lys Phe
Asp Thr Trp Arg Gln Ser Ala Gly Arg Leu.

Preptin is encoded by polynucleotides having the following nucleotide sequences:

| | |
|---|---|
| gacgtgtcgacccctccgaccgtgcttccggacaacttccccagataccccgtgggcaagttcttccaatatga | (SEQ ID No:33) |
| cacctggaagcagtccacccagcgcctg (human) | |
| gacgtgtctacctctcaggccgtacttccggacgacttccccagataccccgtgggcaagttcttcaaattcgac | (SEQ ID No:34) |
| acctggagacagtccgcgggacgcctg (rat) | |
| gacgtgtctacctctcaggccgtacttccggacgacttccccagataccccgtgggcaagttcttccaatatgac | (SEQ ID No:35) |
| acctggagacagtccgcgggacgcctg (mouse) | |

Preptin and analogs thereof may be generated by synthetic or recombinant means (i.e., single or fusion polypeptides). Polypeptides, especially shorter polypeptides up to about 50 amino acids, are conveniently made by chemical synthesis. See, for example, Atherton and Sheppard, Solid Phase Peptide Synthesis: A Practical Approach, New York: IRL Press, 1989; Stewart and Young: Solid-Phase Peptide Synthesis 2nd Ed., Rockford, Ill.: Pierce Chemical Co., 1984; and Jones, The Chemical Synthesis of Peptides, Oxford: Clarendon Press, 1994. For example, to be prepared synthetically, preptin may be synthesized using any of the commercially available solid phase techniques such as the Merrifield solid phase synthesis method, where amino acids are sequentially added to a growing amino acid chain (see, for example, Merrfield, J. Am. Soc. 85:2146-2149 (1963); Marglin, A. and Merrifield, R. B. Annu. Rev. Biochem. 39:841-66 (1970); and Merrifield R. B. JAMA. 210(7):1247-54 (1969)). Variations of the Merrifield solid phase synthesis, for example Fmoc, may also be used to chemically synthesize preptin and preptin analogs. Equipment for automated synthesis of peptides or polypeptides is also commercially available from suppliers such as Perkin Elmer/Applied Biosystems (Foster City, Calif.) and may be operated according to the manufacturers instructions. Confirmation of the identity of the newly synthesized preptin peptides and preptin analogs may be achieved by amino acid analysis, mass spectroscopy, Edman degradation, or by assessing biological function (i.e., stimulating insulin secretion).

Analogs of preptin and of its encoding polynucleotides are also within the scope of the present invention. Such analogs include functional equivalents of preptin and of the polynucleotides described above. Analogs of preptin may be made by substituting amino acids which do not substantially alter the bioactivity of the preptin analog. Selection of amino acids for substitution can depend on the size, structure, charge, and can be either an amino acid found in nature or synthetic amino acid. Generally, amino acids which have a similar charge (i.e., hydrophobic for hydrophobic) or similar size (i.e., isoleucine for leucine) can be selected for substitution. One or more substitutions can be made in a stepwise fashion or concurrently. Variations in the residues included in the peptide are also both possible and contemplated. For example, it is possible to substitute amino acids in a sequence with equivalent amino acids using conventional techniques. Groups of amino acids known normally to be equivalent are:

(a) Ala, Ser, Thr, Pro, and Gly;

(b) Asn, Asp, Glu, and Gln;

(c) His, Arg, and Lys;

(d) Met, Glu, Ile, and Val; and

(e) Phe, Tyr, and Trp

It is understood that many preptin analogs can be achieved by substituting one or more amino acids. The preptin analogs can be tested for biological function (i.e., stimulate insulin secretion in vivo or ex vivo). The biological activity of a preptin analog is at least about 25% of preptin, preferably at least about 35%, preferably at least about 50%, preferably at least about 60%, preferably at least about 75%, preferably at least about 85%, and more preferably at least about 95%.

The invention also encompasses active fragments with preptin bioactive functionality. Such active fragments may be obtained by deletion of one or more amino acid residues of full-length preptin. Active fragments or portions of preptin may be ascertained by stepwise deletions of amino acid residues, from the N-terminal end or the C-terminal end or from within the preptin peptide. If an amino acid is deleted and the bioactivity of preptin is not substantially reduced, then the amino acid may not comprise a portion of the active fragment. Further, polypeptides comprising an active fragment of preptin or preptin analog(s) are also encompassed in the invention.

The invention also encompasses polynucleotides which code for preptin or an active fragment of preptin. Polynucleotides which code for preptin analogs or active fragments of preptin analogs are also encompassed within the invention.

Preptin may also be produced recombinantly by inserting a polynucleotide (usually DNA) sequence that encodes the protein into an expression vector and expressing the peptide in an appropriate host. A polynucleotide encoding the desired polypeptide, whether in fused or mature form, and whether or not containing a signal sequence to permit secretion, may be ligated into expression vectors suitable for any convenient host. Any of a variety of expression vectors (either eukaryotic or prokaryotic) known to those of ordinary skill in the art may be employed. Expression may be achieved in any appropriate host cell that has been transformed or transfected with an expression vector containing a DNA molecule which encodes the recombinant peptides. Suitable host cells include prokaryotes, yeasts and eukaryotic cells. Examples of prokaryotic host cells are known in the art and include, for example, E. coli and B. subtilis. Examples of eukaryotic host cells are known in the art and include yeast, avian, insect, plant, and animal cells such as COS7, HeLa, CHO and other mammalian cells. Standard techniques for recombinant production are described for example, in Maniatis et al. Molecular Cloning—A Laboratory Manual, Cold Spring Harbour Laboratories, Cold Spring Harbour, New York (1989).

The polypeptide is then isolated from lysed cells or from the culture medium and purified to the extent needed for its intended use. Purification or isolation of the polypeptides expressed in host systems can be accomplished by any method known in the art. For example, cDNA encoding a polypeptide intact or a fragment thereof can be operatively linked to a suitable promoter, inserted into an expression vector, and transfected into a suitable host cell. The host cell is then cultured under conditions that allow transcription and translation to occur, and the desired polypeptide is recovered. Other controlling transcription or translation segments, such as signal sequences that direct the polypeptide to a specific cell compartment (i.e., for secretion), can also be used.

A fusion protein may also be constructed that facilitates purification. Examples of components for these fusion proteins include, but are not limited to myc, HA, FLAG, His-6, glutathione S-transferase, maltose binding protein or the Fc portion of immunoglobulin. These methods are known in the art. See, for example, Redd et al. (1997) J. Biol. Chem. 272:11193-11197.

Preferably, especially if used for diagnostic purposes, the polypeptides are at least partially purified or isolated from other cellular constituents. Preferably, the polypeptides are at least about 50% pure. In this context, purity is calculated as a weight percent of the total protein content of the preparation. More preferably, the proteins are 50-75% pure. More highly purified polypeptides may also be obtained and are encompassed by the present invention. The polypeptides are preferably highly purified, at least about 80% pure, and free of pyrogens and other contaminants. Methods of protein purification are known in the art and are not described in detail herein.

Host Cells Comprising Preptin Polynucleotides

Another embodiment of this invention are host cells transformed or transfected with (i.e., comprising) polynucleotides coding for preptin or preptin analogs, polynucleotides coding for active fragments of preptin or active fragments of preptin analogs, vectors comprising polynucleotides coding for active fragments of preptin or active fragments of preptin analogs, or other vectors as described above. Both prokaryotic and eukaryotic host cells may be used. Prokaryotic hosts include bacterial cells, for example E. coli and B. subtilis. Among eukaryotic hosts are yeast, insect, avian, plant and mammalian cells. One example of a mammalian host cell is NS0, obtainable from the European Collection of Cell Cultures (England). Transfection of NS0 cells with a plasmid, for example, which is driven by a cytomegalovirus (CMV) promoter, followed by amplification of this plasmid in using glutamine synthetase provides a useful system for protein production. Cockett et al. Bio/Technology 8:662-667 (1990).

The host cells of this invention can be used, inter alia, as repositories of preptin polynucleotides and/or vehicles for production of preptin polynucleotides and polypeptides. They may also be used as vehicles for in vivo delivery of preptin polypeptides.

Isolation of Preptin Polynucleotides

Preptin polynucleotides can be isolated from various sources including but not limited to host cells transformed (or transfected) with expression vector expressing preptin, circulating preptin in vivo, biological samples or fluids, tissue samples (i.e., pancreas), or from a cloning vector. Isolation of nucleotides from cells is routine to a skilled artisan and may be achieved using any number of commercially available nucleotide isolation kits, for example from Qiagen (Valencia, Calif.) or Promega (Madison, Wis.). Polynucleotides encoding preptin may be in the form of DNA, RNA, DNA analogs, RNA analogs, or a hybrid of DNA-RNA. Preptin polynucleotides can also be single stranded or double stranded.

In terms of preptin itself, functional equivalents include all proteins that function as preptin and have a minimum of 6 amino acids as disclosed in SEQ ID No:1, SEQ ID No:3, or SEQ ID No:4. Preferably, the functional equivalents have a mimimum of 8 amino acids, more preferably 9, 10, 12, 14, 15, 17, 18, 20, 22, 24, 26, 28, or 30 amino acids. Preferably, these functional equivalents are immunologically cross-reactive. That equivalent may, for example, be a fragment of preptin containing from 6 to 33 amino acids (usually representing a C-terminal truncation) and including a preptin active site or sites, a substitution, addition or deletion mutant of preptin, or a fusion of preptin or a fragment or a mutant with other amino acids.

The six amino acids forming the smallest fragment can be from any part of the sequence, provided they are consecutive in that sequence and fulfil the functional requirement. It is of course also possible (and expressly contemplated) that the bioactive peptide include any one of those hexapeptides, or indeed be or include any heptapeptide, octapeptide, nonapeptide, or decapeptide from the sequence. Peptides which are, or include a hexapeptide, heptapeptide, octapeptide, nonapeptide or decapeptide from human preptin are particularly preferred.

Additions and/or deletions of amino acids may also be made as long as the resulting peptide has substantially the same function as preptin and is preferably immunologically cross-reactive with preptin.

Equivalent polynucleotides include nucleic acid sequences that encode proteins equivalent to preptin as defined above. Equivalent polynucleotides also include nucleic acid sequences that, due to the degeneracy of the nucleic acid code, differ from native polynucleotides in ways that do not effect the corresponding amino acid sequences.

A prediction of whether a particular polynucleotide or polypeptide is equivalent to those given above can be based upon homology. Polynucleotide or polypeptide sequences may be aligned, and percentage of identical nucleotides in a specified region may be determined against another sequence, using computer algorithms that are publicly available. Two exemplary algorithms for aligning and identifying the similarity of polynucleotide sequences are the BLASTN and FASTA algorithms. The similarity of polypeptide sequences may be examined using the BLASTP algorithm. Both the BLASTN and BLASTP software are available on the NCBI anonymous FTP server (ftp://ncbi.nlm.nih.gov) under /blast/executables/. The BLASTN algorithm version 2.0.4 (Feb-24-1998), set to the default parameters described in the documentation and distributed with the algorithm, is preferred for use in the determination of variants according to the present invention. The use of the BLAST family of algorithms, including BLASTN and BLASTP, is described at NCBI's website at URL http://www.ncbi.nlm.nih.gov/BLAST/newblast.html and in the publication of Altschul, Stephen F, et al (1997). “Gapped BLAST and PSI-BLAST: a new generation of protein database search programs”, Nucleic Acids Res. 25:3389-3402. The computer algorithm FASTA is available on the Internet at the ftp site ftp://ftp.virginia.edu.pub/fasta/. Version 2.0u4, February 1996, set to the default parameters described in the documentation and distributed with the algorithm, is preferred for use in the determination of variants according to the present invention. The use of the FASTA algorithm is described in the W R Pearson and D. J. Lipman, “Improved Tools for Biological Sequence Analysis,” Proc. Natl. Acad. Sci. USA 85:2444-2448 (1988) and W. R. Pearson, “Rapid and Sensitive Sequence Comparison with FASTP and FASTA,” Methods in Enzymology 183:63-98 (1990).

Analogs according to the invention also include the homologues of preptin from species other than human, rat or mouse. Such homologues can be readily identified using, for example, nucleic acid probes based upon the conserved regions of the polynucleotides which encode human, rat and mouse preptin.

Preptin or its analogs can also be present in various degrees of purity. Preferably, the preptin/analog component makes up at least 50% by weight of the preparation, more preferably at least 80% by weight, still more preferably at least 90% by weight, still more preferably at least 95% by weight and yet more preferably at least 99% by weight. It is however generally preferred that, for pharmaceutical application, the preptin or analog be present in a pure or substantially pure form.

Administration of Preptin

For administration to a patient, it is possible for preptin or preptin analogs to be used as such pure or substantially pure compounds. However, preptin or preptin analogs may also be presented as a pharmaceutical composition. Such compositions may comprise preptin or preptin analogs together with one or more pharmaceutically acceptable carriers therefor and optionally other therapeutic ingredients where desirable. Formulations for parenteral and nonparenteral drug delivery are known in the art and are set forth in Remington's Pharmaceutical Sciences, 18th Edition, Mack Publishing (1990).

The carrier must be acceptable in the sense of being compatible with the preptin or preptin analog and not deleterious (i.e., harmful) to the patient to be treated. Desirably, the composition should not include substances with which peptides are known to be incompatible. For solid compositions, conventional non-toxic carriers include, for example mannitol, lactose, starch, magnesium stearate, magnesium carbonate, sodium saccharin, talcum, cellulose, glucose, sucrose, pectin, dextrin, tragacanth, methyl cellulose, sodium carboxymethyl cellulose, a low-melting wax, cocoa butter, and the like may be used. The active compound as defined above may be formulated as suppositories using, for example, polyalkylene glycols, for example, propylene glycol as a carrier.

A solid carrier can be one or more substances which may also act as diluents, flavoring agents, solubilizers, lubricants, suspending agents, binders, or tablet disintegrating agents; it can also be an encapsulating material. In a similar manner, cachets or transdermal systems are included. In powders, the carrier is a finely divided solid which is in a mixture with the finely divided active component. In tablets, the active component is mixed with the carrier having the necessary binding properties in suitable proportions and compacted in the shape and size desired.

Liquid form preparations include solutions, suspensions, or emulsions suitable, for example, for oral administration. Aqueous solutions for oral administration can be prepared by dissolving the active compound in water and adding suitable flavorants, coloring agents, stabilizers, and thickening agents as desired. Aqueous suspensions or emulsions for oral use can be made by dispersing the finely divided active component in water together with a viscous material such as natural or synthetic gums, resins, methyl cellulose, sodium carboxymethylcellulose, and other suspending agents known to the pharmaceutical formulation art. If desired, the pharmaceutical composition to be administered may also contain minor amounts of non-toxic auxiliary pH buffering agents and the like, for example, sodium acetate, sorbitan monolaurate, triethanolamine oleate, etc. Actual methods of preparing such dosage forms are known, or will be apparent to those skilled in this art; for example, see Remington's Pharmaceutical Sciences. The composition or formulation to be administered will preferably contain a quantity of the active compound in an amount effective to stimulate the levels of insulin in the individual being treated.

The dosage range is from 0.01 mmol/kg/day to 100 mmol/kg/day, more preferably 0.025 mmol/kg/day to 50 mmol/kg/day, more preferably from 0.05 nmol/kg/day to 25 mmol/kg/day, and more preferably from 0.1 mmol/kg/day to 10 mmol/kg/day. It is understood that the dosage administered may vary from individual to individual. It is also understood that the dosage may be administered in a single dose or optionally over multiple doses (i.e., up to four doses per day). A clinician or physician will routinely be able to determine the dosage needed for individuals. A clinician or physician may monitor factors including but not limited to glucose level, preptin level (either circulating or resident in tissues), insulin levels (either circulating or resident in tissues), level of depletion of pancreatic β-cells, presence or absence of polydipsia, presence or absence of polyphagia, presence or absence of polyuria, levels of glycated hemoglobin, levels of glycated albumin, and levels of fructosamine.

The compositions may conveniently be presented in unit dosage form and may be prepared by any of the methods well known in the art of pharmacy. All methods include the step of bringing the active ingredients into association with a carrier which constitutes one or more accessory ingredients.

The precise form the composition will take will largely be dependent upon the administration route chosen. For example, preptin or preptin analogs may be injected parenterally, e.g. intravenously into the blood stream of the patient being treated. However, it will be readily appreciated by those skilled in the art that the route can vary, and can be intravenous, subcutaneous, transcutaneously, intramuscular, intraperitoneal, enterally, transdermally, transmucously, sustained release polymer compositions (e.g. a lactide polymer or co-polymer microparticle or implant), perfusion, pulmonary (e.g., inhalation), nasal, oral, etc. Injectables can be prepared in conventional forms, either as liquid solutions or suspension, solid forms suitable for solution or suspension in liquid prior to injection, or as emulsions. Suitable excipients include, for example, water, saline, aqueous dextrose, glycerol, ethanol or the like. In addition, if desired, the pharmaceutical compositions may also contain minor amounts of non-toxic substances such as wetting or emulsifying agents, auxiliary pH buffering agents and the like, for example, sodium acetate, sorbitan monolaurate, triethanolamine oleate, etc.

Compositions suitable for parenteral and in particular subcutaneous administration are preferred. Other suitable administration routes are intravenous administration and intramuscular administration. Such compositions conveniently comprise sterile aqueous solutions of preptin or the preptin analog. Preferably, the solutions are isotonic with the blood of the patient to be treated. Such compositions may be conveniently prepared by dissolving the preptin or analog in water to produce an aqueous solution and rendering this solution sterile. The composition may then be presented in unit or multi-dose containers, for example sealed ampoules or vials.

One particularly preferred composition is preptin in a physiological buffer solution suitable for injection.

Compositions suitable for sustained release parenteral administrations (eg. biodegradable polymer formulations) are also well known in the art. See, for example, U.S. Pat. Nos. 3,773,919 and 4,767,628 and PCT Publication No. WO 94/15587.

It is also convenient for preptin to be converted to be in the form of a salt. Such a salt will generally be physiologically acceptable, and can be formed using any convenient art standard approach.

Preptin salts formed by combination of preptin with anions of organic acids are particularly preferred. Such salts include, but are not limited to, malate, acetate, propionate, butyrate, oxaloacetate, citrate, isocitrate, α-ketoglutarate, succinate, fumarate and trifluoroacetate salts.

The salts this formed can also be formulated into pharmaceutical compositions for therapeutic administration where this is desired.

Methods for Using Preptin Polynucleotides

As described above, the present invention provides preptin (including in its human, rat and mouse forms) and analogs of preptin. Preptin and its analogs play a physiological role in the stimulation of glucose evoked insulin secretion.

The invention therefore also provides methods by which glucose-evoked insulin secretion can be modulated. Such modulation will usually involve administration of preptin and its analogs as described above. However, modulation can also be achieved by use of preptin agonists and antagonists.

A preptin agonist is a compound which promotes or potentiates the effect of preptin on insulin secretion. In contrast, a preptin antagonist is a compound which competes with preptin or otherwise interacts with preptin to block or reduce the effect of preptin on insulin secretion.

Preptin agonists and preptin antagonists can be identified by assay systems which measure the effect preptin has on insulin secretion in the presence and absence of a test compound. For example, the assay systems described in the experimental section herein can be used.

Where it is desired that a preptin agonist or preptin antagonist be employed in modulating insulin secretion, the agonist/antagonist can be administered as a pure compound or formulated as a pharmaceutical composition as described above for preptin.

The polynucleotides of this invention have several uses. Preptin polynucleotides are useful, for example, in expression systems for the recombinant production of preptin or preptin fragments. They are also useful as hybridization probes to assay for the presence of preptin polynucleotide (or related) sequences in a sample using methods well known to those in the art. Further, preptin polynucleotides are also useful as primers to effect amplification of desired polynucleotides. The polynucleotides of this invention may be also useful as vaccines and for gene therapy.

Preptin polynucleotides of this invention can be used as primers for amplification of polynucleotides encoding preptin or a fragment thereof, such as in a polymerase chain reaction (PCR). Further, the preptin polynucleotides can be also used as PCR primers to screen for or to detect other genes associated with preptin or genes related to disease or disease states which are also associated with preptin. The conditions for carrying out PCR reactions depend on the specificity desired, which in turn can be adjusted by the primer used and the reaction conditions. Such adjustments are known in the art and need not be discussed in detail herein.

Preptin polynucleotides can also be used as hybridization probes for detection of, for example, the presence of preptin polynucleotides in a cell. For instance, a preptin polynucleotide could be used as a probe to determine the presence of preptin polynucleotide sequences in cells used in gene therapy. For these methods, suitable cells from a biological sample or a sample derived from cells (either of which are suspected of containing preptin polynucleotide sequences) is obtained and tested for the presence of preptin polynucleotide by contacting the polynucleotides from the sample with the preptin polynucleotide probe. The method is conducted to allow hybridization to occur between the preptin probe and preptin polynucleotide of interest, and the resultant (if any) hybridized complex is detected. Such methods entail procedures well known in the art, such as cell culture, polynucleotide preparation, hybridization, and detection of hybrid complexes formed, if any. Using similar methods, the probes can also be used to detect vectors which are in turn used to produce preptin polypeptides, intact preptin, or recombinant, variant forms of preptin.

The preptin polynucleotides of this invention can be used in expression systems to produce preptin polypeptides, intact preptin, or recombinant forms of preptin, including intact preptin, which have enhanced, equivalent, or different, desirable properties. These recombinant forms are made by using methods disclosed supra or other routine methods in the art. Examples of recombinant forms of preptin and preptin polypeptides include, but are not limited to, hybrids, chimeras, single chain variants, and fusion proteins containing other components such as cytokines.

Other uses of preptin polynucleotides or polypeptides may be for vaccines and gene therapy. One general principle is to administer the polynucleotide so that it either promotes or attenuates the expression of the polypeptide encoded therein. Thus, the present invention includes methods of inducing a glucose-evoked insulin response and methods of treatment comprising administration of an effective amount of preptin polynucleotide(s) to an individual. In these methods, a preptin polynucleotide encoding a preptin polypeptide is administered to an individual, either directly or via cells transfected with the preptin polynucleotide(s). Preferably, the preptin polynucleotide is replicated inside a cell. Thus, the preptin polynucleotide(s) is operatively linked to a suitable promoter, such as a heterologous promoter that is intrinsically active in cells of the target tissue type. Entry of the polynucleotide into the cell is accomplished by techniques known in the art, such as via a viral expression vector, such as a vaccinia or adenovirus vector, or association of the polynucleotide with a cationic liposome. Preferably, the preptin polynucleotide(s) are in the form of a circular plasmid, preferably in a supercoiled configuration. Preferably, once in cell nuclei, plasmids persist as circular non-replicating episomal molecules.

Another use for preptin polypeptides is for generation of antibodies, including monoclonal antibodies. Preptin polypeptides are used as immunogens to immunize mice. Splenocytes (including lymphocytes) are obtained from the immunized mice. Hybridomas are prepared from the lymphocytes and immortalized myeloma cells using the general somatic cell hybridization technique of Kohler, B. and Milstein, C. Nature 256:495-497 (1975). Other modified methods, for example by Buck, D. W., et al., In Vitro, 18:377-381 (1982) may also be used. Available myeloma lines, including but not limited to X63-Ag8.653 and those from the Salk Institute, Cell Distribution Center, San Diego, Calif., USA, may be used in the hybridization. The technique involves fusing the myeloma cells and lymphoid cells using a fusogen such as polyethylene glycol, or by electrical means well known to those skilled in the art. After the fusion, the cells are separated from the fusion medium and grown in a selective growth medium, such as HAT medium, to eliminate unhybridized parent cells. Any of the media described herein, supplemented with or without serum, can be used for culturing hybridomas that secrete monoclonal antibodies. As another alternative to the cell fusion technique, EBV immortalized B cells are used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbants made of the immunogen attached to a solid phase and eluting or releasing the desired antibodies off the immunogen.

Preptin polypeptides may also be used as immunogens to immunize other animals (i.e., rats and rabbits) to generate polyclonal antibodies. Methods of producing polyclonal antibodies and isolation and purification thereof is known in the art. See, for example, Harlow and Lane (1987). Other suitable techniques for preparing antibodies involve in vitro exposure of lymphocytes to the antigen or alternatively to selection of libraries of antibodies in phage or similar vectors. See, for example Huse et al., 1989.

Also, recombinant antibodies may be produced using procedures known in the art. See, for example, U.S. Pat. No. 4,816,567.

The antibodies may be used with or without modification. Frequently, antibodies will be labeled by joining, either covalently or non-covalently a substance which provides a detectable signal. A wide variety of labels and conjugation techniques are known and are reported extensively in the literature.

Antibodies as above to preptin can therefore be used to monitor the presence of preptin in a patient or in preptin quantification assays. Further, anti-preptin antibodies can be used to measure levels of preptin in an individual, either at one fixed time point or over a period of time to monitor fluctations in circulating preptin levels. Anti-preptin antibodies can also be used to measure levels of preptin in an individual to whom drugs have been administered. In such assays, any convenient immunological format can be employed. Such formats include immunohistochemical assays, RIA, IRMA and ELISA assays.

The assays can be conducted in relation to any biological fluid which does, or should, contain preptin. Such fluids include blood, serum, plasma, urine and cerebrospinal fluid. Antibodies, monoclonal or polyclonal, against preptin may be used for diagnosis or for therapeutic purposes. Antibodies may be used by themselves or attached to a solid substrate, such a column or a plate. Antibodies which are attached to a solid substrate may be used for assays, for example ELISA, or as a standard in other assays. Antibodies against preptin are also useful for preptin isolation, preptin purification, and preptin quantitation.

The antibodies can also be included in assay kits. Such kits can contain, in addition, a number of optional but conventional components, the selection of which will be routine to the art skilled worker. Such additional components will however generally include a preptin reference standard, which may be preptin itself or an analog (such as a fragment).

It will also be appreciated that antibodies such as described above can, if some circumstances also function as preptin antagonists by binding to preptin and partly or completely interfering with preptin activity.

As alluded to above, the applicants findings with respect of preptin also have diagnostic implications. For example, individuals whose preptin production is less than is required in order to elicit insulin secretion at appropriate levels, or who produce preptin in a less active or inactive (mutant) form will require therapeutic intervention. Diagnostic or prognostic methods are therefore within the scope of the invention.

In one specific embodiment, a diagnostic or prognostic method will involve detection of mutations in the gene coding for preptin and/or the preptin secretory mechanism. Detection can occur using any one of a number of art standard techniques including Single Stranded Confirmation Analysis (Orita et al. (1989)) or the Amplification Refractory Mutation System (ARMS) as disclosed in European Patent Application Publication No. 0 332 435.

If a mutation is detected, corrective approaches become possible. These include but are not limited to gene therapy. Again, art standard techniques will be employed.

Other implications and applications of the applicants identification of preptin will be apparent to those persons skilled in the art, who will appreciate that the above description is provided by way of example only and that the invention is not limited thereto.

Aspects of the invention will now be described with reference to the following non-limiting examples.

### EXAMPLES

### Example 1

Methods and Materials

Cell Culture

βTC6-F7 murine pancreatic islet β-cells, passages 49-60, were cultured at 37° C. in O₂:CO₂95:5 (v/v) in triple flasks in DMEM (Gibco) supplemented with 15% heat-inactivated horse serum and 2.5% fetal bovine serum, and subcultured every 5 d by washing with PBS followed by trypsinization (2.5% Trypsin-EDTA). Each flask yielded approximately 2.0×10⁸ cells at 70% confluence.

Granule purification

β-cells at passages 55-60 from 8-12 triple flasks were harvested by trypsinization, yielding on average 2.5-4.0 ml of pure cells (1.6-2.4×10⁹), which then were concentrated (1700×g, 5 min), washed twice with PBS, and once with Homogenization Medium (0.3 M sucrose/10 mM MES K (Sigma)/1 mM K₂EGTA/1 mM Mg₂SO₄/pH 6.5). then homogenized on ice in the same medium at 1:5 (v/v). The cell suspension was homogenized by 20 passages through a ball-bearing homogenizer (7.87×cm clearance), then clarified by centrifugation (400×g. 10 min), the pellet once re-homogenized and centrifuged, and the supernatants combined (final vol=20 ml). Solutions (v/v) of 13% and 31% OptiPrep™ (Nycomed) were prepared by dilution with Homogenization Medium. and 6×10 ml continuous gradients (31%-13% OptiPrep) poured (Auto Densi-Flow II, Haakebuchler) into Ultra-Clear tubes (Beckman). Pelleted material was over-layered or under-layered, then ultracentrifuged (SW40 Ti/160,000×g/16 h/4° C.). Fractions with RI of 1.363-1,368, containing highest purity secretory granules, were collected, whereas mitochondria and lysosomes were isolated to fractions with RI>1.371. Integrity of granule preparations was monitored using radioimmunoassays for insulin (crystalline granule core), amylin (granule matrix); purity by functional assays for aryl sulphatase (lysosomes) and citrate synthase (mitochondria): and total protein content using Bicinchoninic acid (Pierce).

RP-HPLC

Granule proteins were purified in two sequential RP-HPLC runs [A: 0.08% TFA v/v; B: 80% acetonitrile with 20% A: Applied Biosystems 140B/785A/112A system; Jupiter C18 RP column, 250×2.0 mm (Phenomonex); 250-300 μl/min; A₂₁₄). Secretory granule material was initially centrifuged (16,000×g. 20 min) before loading. An initial 15 min isocratic step was employed, and sequential 30s fractions collected from 19 min post-injection. Slightly different gradients were used sequentially to purify proteins: the first semi-purified granule proteins, whereas the second was slightly flatter, to increase resolution and purity.

Peptide Sequence Analysis

Purified peptides were identified by N-terminal sequence determination (automated Edman method; ABI Procise™) combined with accurate mass determination by MALDI-TOF MS. For complete sequence verification, purified mouse preptin isolates were cleaved using Lys-C (Boehringer Mannheim), and the resulting peptide fragments repurified by RP-HPLC.

MALDI-TOF Mass Spectrometry

Peptide molecular weights were determined by MALDI-TOF MS (Hewlett-Packard G2025A: 337 nm-emission nitrogen laser/150 μJ maximum output/3 ns pulsewidth/30 kV ion acceleration potential) fitted with a 500 MHz digital oscilloscope (G2030AA, LeCroy) using an α-CHC matrix with recombinant human insulin (Novo Nordisk: M+H⁺, 5808.66 Da; M+2H⁺, 2904.83) and somatostatin Bachem; M+H⁺, 1638.91, M+Na⁺, 1660.90) mass standards. MS was performed under high vacuum (<1.0 μTorr) and data acquired (ChemStation, 0-20PS method positive polarity in the 0-20 kDa range) with external mass calibration in “single shots” mode. Accurate molecular weights of purified peptides were confirmed by interpolation with external mass standardization.

Chemical Synthesis of Rat Preptin

The sequences of rat and human preptin were determined by comparison with known predicted IGF-II sequences. Rat preptin was chemically synthesized (Auspep Pty, Australia), according to the predicted sequence, using Fmoc chemistry on an Advanced Chem Tech 396 Robotics Peptide Synthesiser starting with FmocLeu-Wang resin. The peptide was deprotected and cleaved from the resin with a solution 92.5% TFA: 2.5% water: 2.5% trilsopropylsilane: 2.5% dithiothreitol for 3 h. The peptide was precipitated from the TFA solution by addition of diisopropyl ether and the precipitate dissolved in 30% acetonitrile: water, lyophilised, and purified by RP-HPLC. Purity was confirmed as >99% by analytical RP-HPLC (rat preptin eluted at 47%B), while MALDI-TOF MS validated the mass as 3932.4 Da±0.026%.

Preptin Radioimmunoassay

Synthetic rat preptin was conjugated to the carrier, ovalbumin, using the single step glutaraldehyde method at pH 7.0, then used to raise polyclonal antisera in NZW rabbits. Preptin was ¹²⁵I-radiolabelled using the chloramine-T method, and [¹²⁵I]preptin (362 μCi/μg) purified by Sephadex G-10 chromatography (50 mM phosphate buffer, pH 7.5). An optimized RIA for preptin was then developed, with B/F separation by the PEG-assisted second antibody (goat-anti-rabbit method). This employed a dilution of antiserum at 1:10,000 (final assay dilution 1:30,000); tracer at 8,000 cpm/tube; incubation times of 24 h+72 h; and had an EC₂₀ value of 344 pM preptin; EC₈₀ of 39 pM; minimum detectable concentration of 11.2±9.8 pM. at an R/T value of 0:30; and zero cross-reactivity with rodent (rat/mouse) insulins, amylin, and human IGF-II.

Cellular Preptin Secretion

Preptin secretion was studied in βTC6-F7 cells (passage #52), cultured otherwise as above in 24-well plates at 4×10⁵ cells per well. Preptin stimulation was performed after 3 d growth, at 80% confluence. Cells were washed twice in HEPES-buffered KRB before commencement of secretion studies, then preincubatcd for 1 h in 1 ml/well incubation buffer (0 mM glucose; 0.1% w/w Fraction V BSA (Sigma) dissolved in HEPES-KRB) 500 μl/well was then removed, and replaced with an equivalent volume of fresh incubation buffer containing various concentrations of glucose. After 2 h incubation (37° C.), incubation medium was removed, cells washed thrice with PBS, then lysed with lysis buffer. Incubation supernatants and cell lysates were then assayed for insulin and preptin contents using the described RIAs. In separate experiments, time-dependent hormone secretion was also determined.

Characterization of Secreted Preptin-Like Immunoreactive Material (PLIM)

Since preptin is a cleavage product of the E-peptide of IGF-II, and other cleavage products from a similar region have been isolated from serum in the past. (Hylka (1985); Daughaday (1992); Liu (1993)), quantitation by preptin RIA was insufficient to characterize the nature of the secreted and circulating peptide. A combined RP-HPLC/preptin RIA method was therefore developed to further characterize PLIM.2 ml aliquots of separated plasma from a human donor, and βTC6-F7 conditioned medium, were acidified with 0.1 ml of 4M acetic acid and applied to a C-18 Sep Pak (Waters, 1 ml volume) which had been pre-equilibrated with 10 ml of 100% methanol and 20 ml of 4% (v/v) acetic acid. The Sep Pak was washed with 20 ml of 4% acetic acid, before bound components were eluted with 2 ml of 0.1 M acetic acid in 70% methanol, and the final volume of eluate reduced to 150 μl by rotary evaporation. Eluates were then subjected to RP-HPLC as above, and corresponding fractions combined from multiple runs. Fractions likely to contain preptin and insulin were subjected to MALDT-TOF MS. All fractions were then made up to a volume of 350 μl with preptin assay buffer, then analyzed by preptin and insulin RIAs. In order to compare profiles of immunoreactivity of these secreted products with the intragranular profile (FIG. 1b), 10 μl samples from the initial RP-HPLC granule fractions were diluted to a final volume of 610 μl with preptin RIA buffer, and also assayed for insulin and preptin.

Rate of Carbohydrate Metabolism in Isolated Rat Skeletal Muscle

The β-cell hormones amylin and insulin modulate carbohydrate utilization in peripheral tissues, including skeletal muscle. The ability of preptin to alter glucose uptake and incorporation into muscle glycogen was investigated using isolated incubated stripped soleus muscle as a model tissue. All animal methods were carried out with appropriate permission from the Institutional Animal Ethics Committee. Male Wistar rats (200±20 g) were housed in controlled conditions (20° C., 12 h light/dark cycle) and fed standard rat chow (Diet 86, NRM Tegel, Auckland) and water ad libitum. 18-h fasted rats were anesthetized (45 mg/kg Pentobarbitone sodium) then sacrificed by cervical dislocation and soleus muscles dissected under carboxygenated-KHB (O₂:CO₂95: 5 v/v), then incubated in nDMEM supplemented with various concentrations of insulin and preptin. Muscles were teased longitudinally into 3 equal strips with a final radius of approximately 1.5 mm [(U) ¹⁴C] D(+)-glucose (1 mCi/ml, Amersham) was diluted 1:20 (v/v) in 70% ethanol to yield a final concentration of 0.5 μCi/10 μl. Actrapide® Recombinant Human Insulin (100 U/ml, Nova Nordisk) was diluted 1/1000 in 10 ml nDMEM. 60 μg rat preptin was dissolved in 1526 μl of NDMEM to a concentration of 10 μM, then further diluted in NDMEM to give stock solutions of 1 pM, 100 nM, 10 nM, 100 pM and 1 pM. Two different experimental paradigms were employed to determine whether preptin (i) stimulated the rate of glucose incorporation into glycogen, or (ii) acted as an 15 antagonist of insulin-evoked glucose incorporation into glycogen.

Preptin Antagonist Incubation Protocol

Four muscle strips were transferred into each of 9 flasks, which contained 10 ml of carboxygenated NDMEM, 0 (control) or maximally-effective insulin (23.7 nM), and various concentrations of preptin (10 fM, 100 fM, 1 pM. 0, 10 pM, 100 pM, 1 nM or 10 nM). Flasks were then equilibrated in a shaking water bath 30° C., 20 min), following which 10 μl of (0.5 μCi) D-[(U)¹⁴C] glucose was added, at strict 1.5 min intervals. Muscle strips were then incubated for 120 min at 30° C. under carbogen. After incubation, strips were removed from each flask at 1.5 min intervals, and blotted dry. They were then snap-frozen in liquid N₂, freeze-dried for 24 h in pre-weighed tubes, then strip dry-weights determined. Muscle strips were then solubilized in 250 μl or 60% KOH, incubated at 70° C. for 45 min, then cooled before overnight precipitation at −20° C. with ice-cold ethanol. Glycogen pellets were then prepared by centrifugation (9,000×g, 15 min, 0° C.), pellets resuspended, and re-precipitated twice, before the supernatant was finally aspirated and glycogen pellets over-dried at 70° C. for 2 h. incorporation of ¹⁴C was then determined by scintillation counting.

Preptin Agonist Protocol

All methods were as described above, except that strips were incubated in the absence of insulin (except for the positive control, at 23.7 nM) and final preptin concentrations of 0, 0.1, 1, 10 and 100 μM.

Effect of Preptin on Insulin Secretion

Insulin and amylin are known to modulate β-cell insulin secretion via presumed autocrine mechanisms. The effect of preptin on insulin secretion was therefore tested using a β-cell secretagogue protocol βTC6-F7 cells were subcultured at passage #52 into 24-well plates at 4×10⁵ cells/well. They were grown for 3d in nDMEM to 80% confluence, then washed twice with KRB-HEPES. Stock preptin was serially diluted in incubation medium containing 10 mM D(+) glucose to yield final concentrations of 150, 75, 25, 5, 1 and 0.1 nM. Cells were then washed, and 1 ml/well of incubation medium containing 10 mM and-various final preptin concentrations was added to each well. Cells were incubated at 37° C. for 2 h, then medium removed. Cells were washed thrice with PBS, then lysed with 500 μl of lysis buffer. Incubation medium was centrifuged (16,000×g, 3 min) and the supernatant separated from pelleted debris. Incubation medium and lysates were then analyzed for insulin, preptin and protein as above.

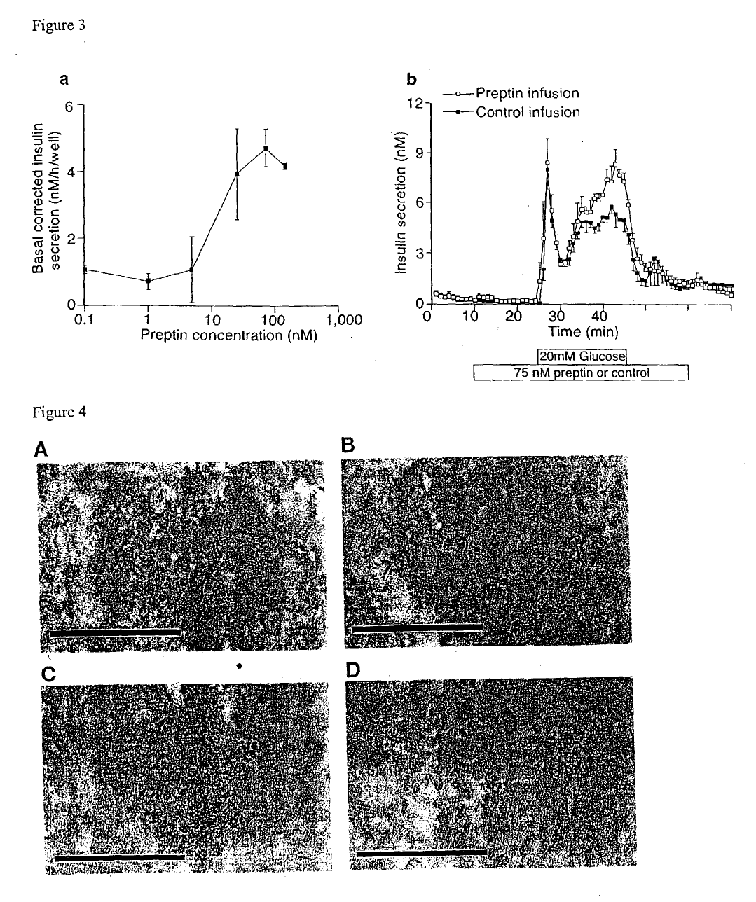

The results of the above are shown in FIG. 3a.

Effects of Preptin and of Anti-Preptin Immunoglobulin on Insulin Secretion from Isolated-Perfused rat Pancreas

Pancreases from fasted adult male Wistar rats (300±25 g) were isolated and prepared (Grodsky and Fanska 1975). Pancreases were perfused with KHB supplemented with 4% dextran, 0.5% BSA, 3 mM arginine and 5.5 mM glucose. Perfusate was gassed with 95% 02:5% CO₂ and infused by peristaltic pump (2.7 ml.min⁻¹ without re-circulation). Pancreases were perfused and equilibrated for 20 min prior to each 70 min perfusion experiment. 10 min into the experiment either carrier buffer (0.1% BSA in 0.9% NaCl), preptin dissolved in carrier buffer, anti-preptin Ig or non-immunised rabbit Ig (both diluted in carrier buffer) were introduced via a side-arm infusion (final concentrations in perfusate: 75 nM preptin; 35 μg.ml⁻¹ Ig). In addition, at 25 min, glucose was infused for 20 min (final concentration in perfusate: 20 mM). 1 min fractions were collected on ice and assayed for insulin (RIA).

The results of the above are shown in FIGS. 3b and 7d.

Mouse preptin is a 34 amino acid peptide which corresponds to Asp₆₉-Leu₁₀₂ of murine proIGF-II E-peptide.

Preptin was present in βTC6-F7 granules at 1:8 the content of insulin, but 2:1 that of amylin (mol/mol), as determined by integration of RP-HPLC peak-areas. Preptin is flanked NH₂-terminally by a recognized Arg cleavage site, and COOH-terminally by a putative dibasic (Arg-Arg) cleavage motif (Bell (1984)) (FIG. 1e). These residues likely serve as post-translational processing signals, and are highly conserved between species. Many prohormone precursors incorporate more than one hormone with differential proteolytic processing often being tissue specific (Martinez (1989)). The above results indicated that proIGF-II is a prohormone with more than one peptide-hormone product.

IGF-II is a member of the insulin family that regulates cell growth, differentiation and metabolism (De Chiara et al (1990). It is a single polypeptide chain derived from the BCA and D domains of proIGF-II (see FIG. 1e) and is widely synthesized in fetal and adult tissues. Insulin expression, on the other hand, is almost completely confined to β-cells. In mammalian genomes, the IGF-II gene is contiguous with those of insulin (Bell (1985)) and recent studies in humans have identified a VNTR polymorphism-upstream of the INS and IGF-II genes, which may contribute to differential regulation of both genes (Ong (1999)).

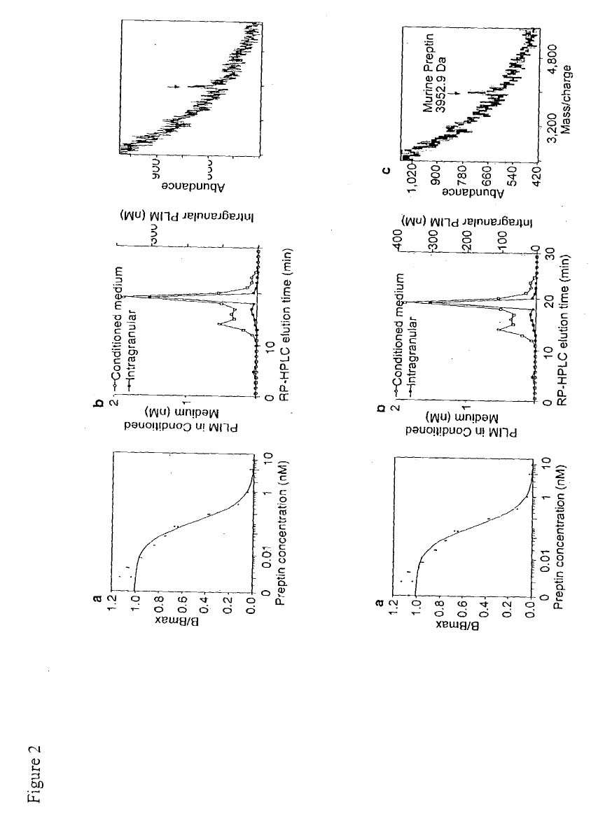

The preptin radioimmunoassay (RIA) (FIG. 2a) and reanalysis of the granule purification profiles of FIGS. 1a with the preptin RIA showed that preptin co-purified with insulin and amylin, confirming that it was indeed a granule component. Preptin-like immunoreactive material (PLIM) was characterized by RP-HPLC/RIA in purified granules and in βTC6-F7 conditioned medium. The major form of both intra-granular and extracellular PLIM co-eluted on RP-HPLC (FIG. 2b). Mass spectrometry of HPLC-purified material corresponding to the PLIM peak from βTC6-F7 cells showed the presence of a single species, with molecular mass identical to that of murine preptin (FIG. 2c). RP-HPLC also demonstrated that the major form of PLIM from human and rat plasma co-eluted with intragranular murine preptin. Preptin was co-secreted with insulin from βTC6-F7 cells in response to glucose-stimulation (FIG. 2d), reaching maximal levels at 1-mM or greater.

These results confirm that preptin is synthesized in islet β-cells and packaged in secretory granules. Further, it is co-secreted with insulin in a glucose-dependent manner.

There is evidence that insulin secretion may be modulated by islet β-cell hormones, including insulin (Kulkami (1999); Elahi (1982); Argoud (1987)), amylin (Waggoner et al (1993); Silvestre (1996); Degano et al (1993)), and pancreastatin (Tatemoto (1986)). These are thought to act through autocrine negative-feedback loops, mediated via binding to specific cell-surface receptors. The effects of preptin on insulin secretion were therefore investigated. The results obtained showed that synthetic rat preptin enhanced the glucose (10-mM)-stimulated secretion of insulin from cultured βTC6-F7 cells, in a manner that was both concentration-dependent and saturable (FIG. 3a). Significant effects of preptin compared to controls (0 added preptin) were detected at concentrations of 0.1-nM and greater, and reached maximal at 75-nM. This concentration is equivalent to that at which amylin elicits inhibition of insulin secretion (Degano et al (1993)). These preptin concentrations are similar to those secreted from βTC6-F7 cells (FIG. 2d), and are thus likely to occur adjacent to β-cell membranes in situ in physiological islets. This demonstration of concentration-dependent and saturable stimulation of insulin secretion by preptin suggests that it elicits these effects by binding to a cell surface receptor.

The effect of infused synthetic rat preptin on glucose (20-mM)-stimulated insulin secretion in the isolated-perfused rat pancrease (FIG. 3b) was measured employing a maximally-effective preptin concentration (75-nM). Preptin significantly enhanced (by 30%; p=0.03. 2-tailed t-test of areas-under-curve) the second phase of insulin secretion, compared with control values (0-added preptin) (FIG. 3b). These findings are consistent with those obtained from βTC6-F7 cells (FIG. 3a). They suggest that preptin is a physiological regulator of insulin secretion, which acts in a newly recognized feed-forward autocrine loop to enhance glucose-stimulated insulin secretion, and may function to counterbalance the inhibitory effects of other β-cell hormones on insulin secretion.

### Example 2

Preptin is Co-Packaged with Insulin in Islet Tissue

To study preptin physiology, competitive immunohistochemical studies were performed in normal murine pancreas using a preptin-specific antiserum. Synthetic rat preptin, prepared as above, (Auspep Pty Ltd) was conjugated to ovalbumin using the single-step gluteraldehyde method at pH 7.0 (Harlow and Lane). New Zealand white rabbits were used to raise polyclonal antisera against the rat preptin conjugate.

Serial sections from normal adult mouse (FVB/n) pancreas were stained with hematoxylin and specific anti-preptin or anti-insulin antisera, all at final dilutions of 1:40 (v/v), and with goat-anti-rabbit immunoperoxidase-labeled second antibody. Preptin (1 or 5 mg.ml−1) was pre-incubated with anti-preptin antiserum for 30 min before addition to sections to demonstrate the specificity of preptin immunostaining.

Preptin-like immunoreactive material (PLIM) and insulin-like immunoreactive material were co-localized in islet β-cells (FIGS. 4a,b). Competition studies showed that PLIM-staining was suppressed by pre-incubating preptin antiserum with synthetic preptin in a concentration dependent manner (FIGS. 4b-d). These studies suggest that preptin is present in physiological pancreatic islet β-cells. PLIM is present in normal islet tissue To establish the identity of PLIM in normal islet tissue, we performed RP-HPLC/RIA of acid ethanol extracts from isolated rat islets. Pancreatic islets were isolated from normal adult male Wistar rats, and the contents extracted with acid ethanol according to a modification of published methods (Wollheim and Sharp (1981), Romanus (1988)). J

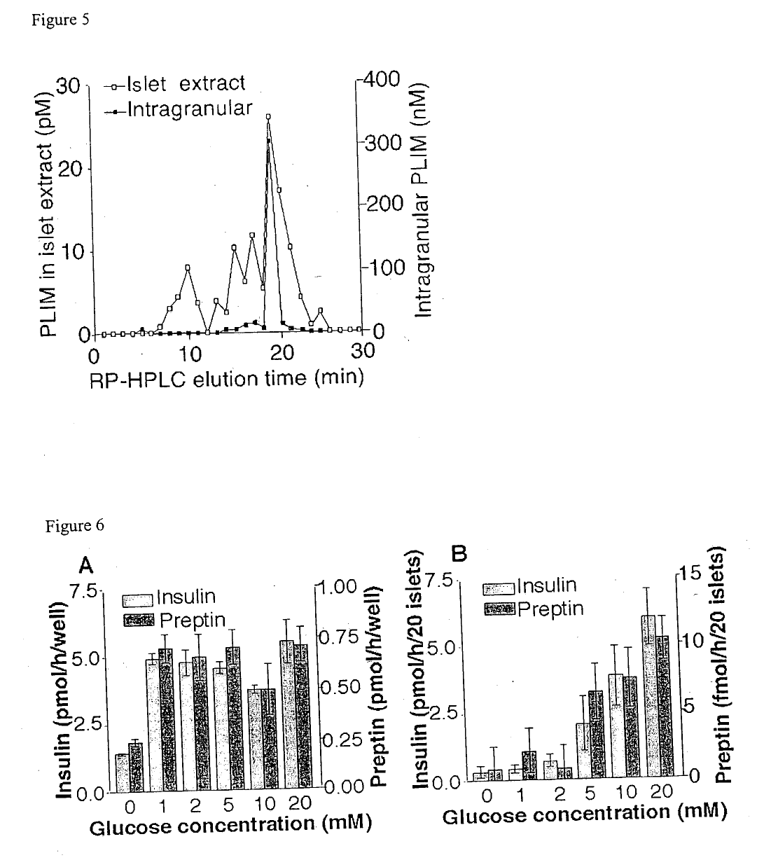

The results are shown in FIG. 5.

Relative to insulin, preptin levels were lower in islet tissue than in βTC6-F7 cells. However, the major peak of PLIM did co-elute with the standard intra-granular preptin, indicating that preptin is the dominant component of PLIM in normal islets (FIG. 3D). The low levels of preptin in rat islet tissue were not unexpected, since adult rats show reduced IGF-II expression and low levels in circulation (H\ddot{oo}g et al., 1993). This is in contrast to humans, who continue to produce high levels of IGF-II throughout their lifetime (Zapf, Walter et al. 1981), from a number of tissues including the pancreas (Bryson et al., 1989; Hoog et al., 1993). These data confirm that the preptin purified from the βTC6-F7 cells was not simply an artifact resulting from proteolysis during purification, but exists and is secreted in this form from both βTC6-F7 cells and normal rat islets.

Preptin is Co-Secreted with Insulin in Response to Glucose Stimulation

Given the co-localization of preptin and insulin within the β-cell secretory granule, experiments were undertaken to determine whether preptin and insulin are co-secreted in a regulated manner. Glucose-stimulated peptide secretion was studied according to published methods using both βTC6-F7 cells (Efrat et al (1993). Knaack et al (1994)) and isolated rat islets (Gotoh et al (1987)), and concentrations of preptin and insulin were measured using specific RIAs.

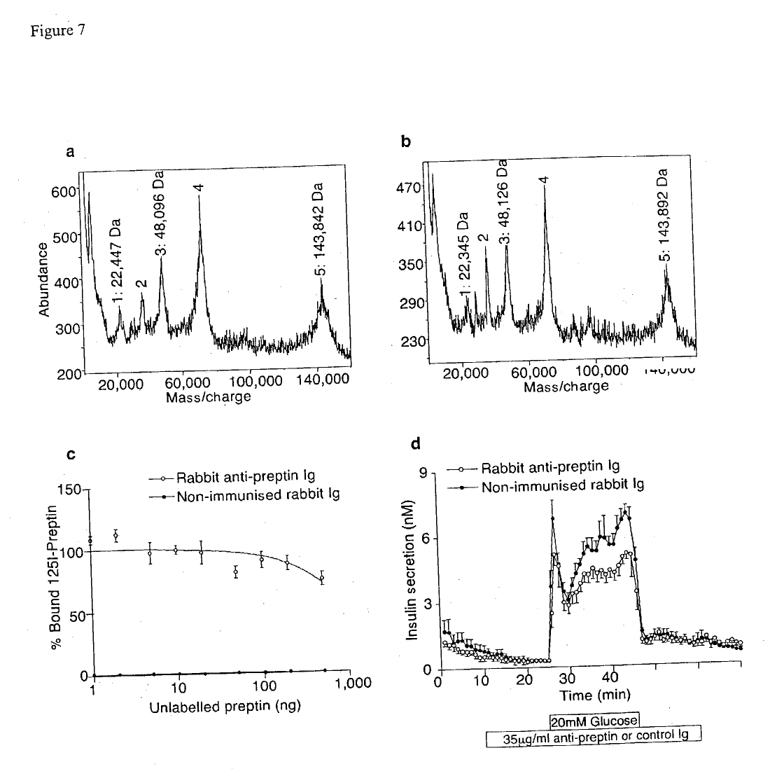

The results are shown in FIG. 6. These indicated that while βTC6-F7 cells were responding to sub-physiological concentrations of glucose (<5 mM (S Efrat, personal communication), a clear pattern of insulin/preptin co-secretion was observed from both βTC6-F7 cells (FIG. 6a) and normal rat islets (FIG. 6b). The amount of preptin secreted from the islet tissue (preptin:insulin 1:500) was much lower than the level secreted from the βTC6-F7 cells (preptin:insulin 1:8). This observation supported the HPLC/RIA results which indicated much lower levels of preptin in physiological tissue than in the cultured β-cells. Although preptin levels are much lower in physiological islets, both of these models clearly showed that preptin is co-secreted with insulin from physiological islet β-cells in response to glucose-stimulation.

Removal of Endogenous Preptin Significantly Decreases Insulin Secretion from the Isolated-Perfused Rat Pancreas

To determine the role that endogenous pancreatic preptin might play in insulin secretion, the action of endogenous preptin was removed by infusing anti-preptin antibodies into the isolated perfused pancreas model as follows:

Pancreases were perfused with KHB supplemented with 4% dextran, 0.5% BSA, 3 mM-arginine and 5.5 mM glucose (final concentrations). Perfusate was gassed with a mixture of 95% 02/5% CO₂ and infused by peristaltic pump at 2.7 ml.min⁻¹ without re-circulation. Pancreases were perfused and equilibrated for 20-min prior to each 70-min perfusion. 10-min into the experiment either anti-preptin γ-globulin or non-immune rabbit γ-globulin were introduced via a side-arm infusion (final γ-globulin concentration in perfusate: 35 μg.ml⁻¹ in carrier buffer (0.1% BSA in 0.9% NaCl). In addition, at 25-min, glucose was infused for 20-min (measured final concentration in perfusate: 20 mM). Continuous 1-min fractions were collected on ice and assayed for insulin (RIA).

Rabbit anti-rat preptin γ-globulin or control (non-immune rabbit) γ-globulin were purified by Protein A affinity chromatography (Pharmacia-Biotech, Hi-Trap Protein A Tech. Rep. (Wikstroms, Sweden (1999)) to diminish the potential influence from other serum constituents. The compositions of the two different γ-globulin fractions were confirmed by MALDI-TOF MS (FIG. 7a, b), and the binding capacities of the two different γ-globulin fractions were determined under conditions simulating the antibody perfusion experiments as above. The maximal amount of preptin completely bound by anti-preptin γ-globulin under the perfused pancreas experimental conditions was 20 ng/min (FIG. 7c).

Isolated perfused pancreases were infused with anti-preptin or control γ-globulin and subjected to square-wave stimulation by 20 mM glucose (FIG. 7d). Secretion of insulin in both the first and second phase was significantly decreased by anti-preptin γ-globulin (first phase: average 29% inhibition compared to controls, P=0.02, 1-tailed t-test; second phase: average 26% inhibition compared to controls, P=0.03, 1-tailed t-test of AUC). In this experiment we have shown that removal of endogenous circulating preptin causes a significant decrease in glucose-mediated insulin secretion. This result is all the more interesting given that preptin has been estimated to be present in relatively low concentrations in the physiological islet(approximately 500× less than insulin) and yet still has the ability to exert a significant effect on insulin secretion. These experiments are consistent with the premise that physiological concentrations of pancreatic preptin play an autocrine role to increase glucose-mediated insulin secretion. This action may be similar to the feed-forward mechanism evoked in platelets by the thrombin-elicited release of thromboxane A₂ (Barrit (1992)).

### REFERENCES

Draznin B. and LeRoith, D. (eds). Molecular biology of diabetes II. Insulin action, effects on gene expression and regulation, and glucose transport (Humana Press Inc., New Jersey (1994)).

Cooper, G. Endocr. Rev. 15, 163-201 (1994).

Waggoner, P., Chen, C., Worley. J., Dukes, I. and Oxford, G. Proc. Natn, Acad. Sci, USA, 90, 9145-9149 (1993).

Hettiarachchi, M., et al. Am. J. Physiol. 273, E859-E867 (1997).

Hutton, J. Diabetologia 12, 271-281 (1989).

Efrat, S. et al. Diabetes. 42, 901-907 (1993).

Knaack, D. et al. Diabetes, 43, 1413-1417 (1994).

Linde, S. et al. J. Chromatgr. 4642, 243-254 (1989).

Johnson, K., et al. Am. J. Pathol. 130, 1-8 (1988).

Bell. G. I., et al. Nature. 310, 775-777 (1984).

Martinez. J. (ed) Peptide hormones prohormones; processing, biological activity, pharmacology (Ellis Horwood Ltd, Chichester, 1989).

DeChiara, T., Ofstradiadis, A. and Robertson, E. Nature 345, 78-89 (1990).

Kulkarni. R. et al. Cell 96, 329-339 (1999).

Elahl, D. et al. N. Eng. J. Med. 306,1196-1202(1982).

Argoud, G., Schade, D. and Eaton. R. Diabetes 36, 959-962 (1987).

Silvestre, R. et al. Br. J. Pharmacol, 117, 347-350 (1996).

Degano, P., Silvestre, R., Salas, M. and Peiro, E. Regul. Pep. 43, 91-96 (1993).

Tatemoto, K. et al. Nature 324, 476-478 (1986).

Barritt, G. Communication within animal cells. (Oxford University Press, Oxford, 1992).

DeFronzo, R., Bonadonna, R. and Ferrannini, E. Diabetes Care 15, 318-368 (1992).

Hutton, J., Penn, E. and Peshavaria, M. Diabetologia 2, 365-373 (1982).

Grodsky, G. and Fanska, R. Methods Enzymol, 39, 364-372 (1975).

Stempien, M., Fong, N., Rall, L. and Bell, G. DNA, 5, 357-361 (1986).

Dull. T., Gray, A. Hayflick, J. and Ullrich, A. Nature 310, 777-780 (1984).

Harlow and Lane (1988). Antibodies: A Laboratory Manual. (Cold Spring Harbour Laboratory, Cold Spring Harbour, New York).

Huse et al. (1989). Science 246: 1275-1281.

Orita et al. (1989). Proc. Natl. Sci., USA, 86: 276-277.

Hylka, V., Teplow, D., Kent, S. and Strauss, D. J. Biol. Chem. 260, 14417-14420 (1985).

Daughaday, W. and Trivedi, B. J. Clin. Endocrinal. Metab.75, 641-645 (1992).

Liu, F., Baker, B., Powell, D. and Hintz, R. J. Clin. Endocrinol. Metabol. 76, 1095-1100 (1993).

Bell, G., Gerhard, D., Fong, N., Sanchez-Pescador, R. and Rall, L. Proc. Natl. Acad. Sci. USA 82, 6450-6454 (1985).

Ong, K. et al. Nat. Genet. 21, 262-263 (1999).

Bryson, J., B. Tuch, et al. (1989). “Production of insulin-like growth factor II by human fetal pancreas in culture.” Journal of Endocrinology 121: 367-373.

Hoog, A., L. Grimelius, et al. (1993). “A high-molecular weight IGF-2 immunoreactive peptide (pro-IGF-2?) in the insulin cells of the islets of Langerhans in pancreas of man and rat.” Regulatory Peptides 47: 275-283.

Zapf, J., H. Walter, et al. (1981). “Radioimmunological determination of insulin like growth factors I and II in normal subjects and in patients with growth disorders and extra pancreatic tumour hypoglycaemia.” Journal of Clinical Investigation 68: 1321-1330.

What is claimed is:

1. An isolated bioactive peptide having preptin functionality.

2. A bioactive peptide, the amino acid sequence of which is as follows:
(SEQ ID No:1)
Asp Val Ser Thr R₁ R₂ R₃ Val Leu Pro Asp R₄ Phe Pro Arg Tyr Pro Val Gly
Lys Phe Phe R₅ R₆ Asp Thr Trp R₇ Gln Ser R₈ R₉ Arg Leu
wherein:
R₁ is Ser or Pro;
R₂ is Gln or Pro;
R₃ is Ala or Thr;
R₄ is Asp or Asn;
R₅ is Gln or Lys;
R₆ is Tyr or Phe;
R₇ is Arg or Lys;
R₈ is Ala or Thr; and
R₉ is Gly or Gln,
or an analog thereof.

3. Isolated human preptin having the amino acid sequence:
(SEQ ID No:2)
Asp Val Ser Thr Pro Pro Thr Val Leu Pro Asp Asn
Phe Pro Arg Tyr Pro Val Gly Lys Phe Phe Gln Tyr
Asp Thr Trp Lys Gln Ser Thr Gln Arg Leu.
or an analog thereof.

4. Rat preptin having the amino acid sequence:
(SEQ ID No:3)
Asp Val Ser Thr Ser Gln Ala Val Leu Pro Asp Asp Phe Pro Arg Tyr Pro Val
Gly Lys Phe Phe Lys Phe Asp Thr Trp Arg Gln Ser Ala Gly Mg Leu.
or an analog thereof.

5. Mouse preptin having the amino acid sequence:
(SEQ ID No:4)
Asp Val Ser Thr Ser Gln Ala Val Leu Pro Asp Asp Phe Pro Arg Tyr Pro Val
Gly Lys Phe Phe Gln Tyr Asp Thr Trp Arg Gln Ser Ala Gly Arg Leu.
or an analog thereof.

6. A mammalian homologue to human, rat or mouse preptin according to any one of claims 3 to 5.

7. A preptin analog which comprises from 6 to 33 amino acids from a sequence according to any one of claims 2 to 5, and which retains preptin functionality.

8. A preptin analog which is, or includes, a hexapeptide, a heptapeptide, an octapeptide, a nonapeptide or a decapeptide derived from human preptin according to claim 3.

9. A peptide selected from human preptin having the amino acid sequence:
(SEQ ID No:2)
Asp Val Ser Thr Pro Pro Thr Val Leu Pro Asp Asn Phe Pro Arg Tyr Pro Val
Gly Lys Phe Phe Gln Tyr Asp Thr Trp Lys Gln Ser Thr Gln Arg Leu.
or an analog thereof, wherein said analog is selected from the following:

| | | |
|---|---|---|
| (i) | Asp Val Ser Thr Pro Pro Thr Val Leu Pro Asp Asn Phe Pro Arg Tyr Pro | (SEQ ID No:5) |
| | Val Gly Lys Phe Phe Gln Tyr Asp Thr Trp Lys Gln Ser Thr Gln Arg; | |
| (ii) | Asp Val Ser Thr Pro Pro Thr Val Leu Pro Asp Asn Phe Pro Arg Tyr Pro | (SEQ ID No:6) |
| | Val Gly Lys Phe Phe Gln Tyr Asp Thr Trp Lys Gln Ser Thr Gln; | |
| (iii) | Asp Val Ser Thr Pro Pro Thr Val Leu Pro Asp Asn Phe Pro Arg Tyr Pro | (SEQ ID No:7) |
| | Val Gly Lys Phe Phe Gln Tyr Asp Thr Trp Lys Gln Ser Thr; | |
| (iv) | Asp Val Ser Thr Pro Pro Thr Val Leu Pro Asp Asn Phe Pro Arg Tyr Pro | (SEQ ID No:8) |
| | Val Gly Lys Phe Phe Gln Tyr Asp Thr Trp Lys Gln Ser; | |
| (v) | Asp Val Ser Thr Pro Pro Thr Val Leu Pro Asp Asn Phe Pro Arg Tyr Pro | (SEQ ID No:9) |
| | Val Gly Lys Phe Phe Gln Tyr Asp Thr Trp Lys Gln; | |
| (vi) | Asp Val Ser Thr Pro Pro Thr Val Leu Pro Asp Asn Phe Pro Arg Tyr Pro | (SEQ ID No:10) |
| | Val Gly Lys Phe Phe Gln Tyr Asp Thr Trp Lys; | |
| (vii) | Asp Val Ser Thr Pro Pro Thr Val Leu Pro Asp Asn Phe Pro Arg Tyr Pro | (SEQ ID No:11) |
| | Val Gly Lys Phe Phe Gln Tyr Asp Thr Trp; | |
| (viii) | Asp Val Ser Thr Pro Pro Thr Val Leu Pro Asp Asn Phe Pro Arg Tyr Pro | (SEQ ID No:12) |
| | Val Gly Lys Phe Phe Gln Tyr Asp Thr; | |
| (ix) | Asp Val Ser Thr Pro Pro Thr Val Leu Pro Asp Asn Phe Pro Arg Tyr Pro | (SEQ ID No:13) |
| | Val Gly Lys Phe Phe Gln Tyr Asp; | |
| (x) | Asp Val Ser Thr Pro Pro Thr Val Leu Pro Asp Asn Phe Pro Aig Tyr Pro | (SEQ ID No:14) |
| | Val Gly Lys Phe Phe Gln Tyr; | |
| (xi) | Asp Val Ser Thr Pro Pro Thr Val Leu Pro Asp Asn Phe Pro Arg Tyr Pro | (SEQ ID No:15) |
| | Val Gly Lys Phe Phe Gln; | |
| (xii) | Asp Val Ser Thr Pro Pro Thr Val Leu Pro Asp Asn Phe Pro Arg Tyr Pro | (SEQ ID No:16) |
| | Val Gly Lys Phe Phe; | |
| (xiii) | Asp Val Ser Thr Pro Pro Thr Val Leu Pro Asp Asn Phe Pro Axg Tyr Pro | (SEQ ID No:17) |
| | Val Gly Lys Phe; | |
| (xiv) | Asp Val Ser Thr Pro Pro Thr Val Leu Pro Asp Asn Phe Pro Arg Tyr Pro | (SEQ ID No:18) |
| | Val Gly Lys; | |
| (xv) | Asp Val Ser Thr Pro Pro Thr Val Leu Pro Asp Asn Phe Pro Arg Tyr Pro | (SEQ ID No:19) |
| | Val Gly; | |
| (xvi) | Asp Val Ser Thr Pro Pro Thr Val Leu Pro Asp Asn Phe Pro Arg Tyr Pro | (SEQ ID No:20) |
| | Val; | |
| (xvii) | Asp Val Ser Thr Pro Pro Thr Val Leu Pro Asp Asn Phe Pro Arg Tyr Pro; | (SEQ ID No:21) |
| (xviii) | Asp Val Ser Thr Pro Pro Thr Val Leu Pro Asp Asn Phe Pro Arg Tyr; | (SEQ ID No:22) |
| (xix) | Asp Val Ser Thr Pro Pro Thr Val Leu Pro Asp Asn Phe Pro Arg; | (SEQ ID No:23) |
| (xx) | Asp Val Ser Tbr Pro Pro Thr Val Leu Pro Asp Asn Phe Pro; | (SEQ ID No:24) |
| (xxi) | Asp Val Ser Thr Pro Pro Thr Val Leu Pro Asp Asn Phe; | (SEQ ID No:25) |
| (xxii) | Asp Val Ser Tbr Pro Pro Thr Val Leu Pro Asp Asn; | (SEQ ID No:26) |
| (xxiii) | Asp Val Ser Thr Pro Pro Thr Val Leu Pro Asp; | (SEQ ID No:27) |
| (xxiv) | Asp Val Ser Thr Pro Pro Thr Val Leu Pro; | (SEQ ID No:28) |
| (xxv) | Asp Val Ser Thr Pro Pro Thr Val Leu; | (SEQ ID No:29) |
| (xxvi) | Asp Val Ser Thr Pro Pro Thr Val; | (SEQ ID No:30) |
| (xxvii) | Asp Val Ser Thr Pro Pro Tbr; and | (SEQ ID No:31) |
| (xxviii) | Asp Val Ser Thr Pro Pro. | (SEQ ID No:32) |

10. An isolated polynucleotide which encodes preptin or an analog thereof according to any one of claims 1-5, 8, and 9.

11. An isolated polynucleotide which encodes preptin or an analog thereof according to claim 6.

12. An isolated polynucleotide which encodes preptin or an analog thereof according to claim 7.

13. An isolated polynucleotide which encodes human preptin and which comprises the following nucleotide sequence:
(SEQ ID No:33)
gacgtgtcgacccctccgaccgtgcttccggacaacttccccagataccccgtgggcaagttettccaatatga
cacctggaagcagtccacccagcgcctg.

14. An isolated polynucleotide which encodes rat preptin and which comprises the following nucleotide sequence:
(SEQ ID No:34)
gacgtgtctacctctcaggccgtacttccggacgacttccccagtaccccgtgggcaagttcttcaaatatcgac
acctggagacagtccgcgggacgcctg.

15. An isolated polynucleotide which encodes mouse preptin and which comprises the following nucleotide sequence:
(SEQ ID No:35)
gacgtgtctacctctcaggccgtacffccggacgacttccccagataccccggggcaagttcttccatatgac
acctggagacagtccgcgggacgcctg.

16. A vector which comprises a polynucleotide having the nucleotide sequence of any one of claims 13 to 15 and which is capable of expressing a peptide having preptin functionality.

17. A vector according to claim 16 which comprises the nucleotide sequence of claim 13.

18. A pharmaceutical composition which comprises preptin or an analog thereof according to any one of claims 1-5, 8, and 9.

19. A pharmaceutical composition which comprises preptin or an analog thereof according to claim 6.

20. A pharmaceutical composition which comprises preptin or an analog thereof according to claim 7.

21. A pharmaceutical composition according to any one of claims 1-5, 8, and 9 further comprising a physiological buffer solution suitable for administration to humans.

22. A pharmaceutical composition according to claim 6 further comprising a physiological buffer solution suitable for administration to humans.

23. A pharmaceutical composition according to claim 7 further comprising a physiological buffer solution suitable for administration to humans.

24. A preparation of mammalian preptin in which said preptin or analog is between 50% and 99% pure.

25. A preparation according to claim 24 in which said preptin or analog is human preptin or an analog thereof.

26. A salt of a mammalian preptin or analog according to any one of claims 1-5, 8, and 9.

27. A salt of a mammalian preptin or analog according to claim 6.

28. A salt of a mammalian preptin or analog according to claim 7.

29. A salt according to claim 26 which is a physiologically acceptable salt.

30. A salt according to claim 27 which is a physiologically acceptable salt.

31. A salt according to claim 28 which is a physiologically acceptable salt.

32. A salt according to claim 29 in which said preptin or analog is formed by combination with anions of an organic acid.

33. A salt according to claim 30 in which said preptin or analog is formed by combination with anions of an organic acid.

34. A salt according to claim 31 in which said preptin or analog is formed by combination with anions of an organic acid.

35. A salt according to claim 32 in which said salt is selected from malate, acetate, propionate, butyrate, oxaloacetate, citrate, isocitrate, α-ketoglutarate, succinate, fumarate and trifluoroacetate salts.

36. A salt according to claim 33 in which said salt is selected from malate, acetate, propionate, butyrate, oxaloacetate, citrate, isocitrate, α-ketoglutarate, succinate, fumarate and trifluoroacetate salts.

37. A salt according to claim 34 in which said salt is selected from malate, acetate, propionate, butyrate, oxaloacetate, citrate, isocitrate, α-ketoglutarate, succinate, fumarate and trifluoroacetate salts.

38. A pharmaceutical composition which includes a salt according to claim 26.

39. A pharmaceutical composition which includes a salt according to claim 27.

40. A pharmaceutical composition which includes a salt according to claim 28.

41. A pharmaceutical composition which includes a salt according to claim 29.

42. A pharmaceutical composition which includes a salt according to claim 30.

43. A pharmaceutical composition which includes a salt according to claim 31.

44. A pharmaceutical composition which includes a salt according to claim 32.

45. A pharmaceutical composition which includes a salt according to claim 33.

46. A pharmaceutical composition which includes a salt according to claim 34.

47. A pharmaceutical composition which includes a salt according to claim 35.

48. A pharmaceutical composition which includes a salt according to claim 36.

49. A pharmaceutical composition which includes a salt according to claim 37.

50. A method of therapeutically or prophylactically treating a patient which comprises the step of administering to said patient an effective amount of preptin or an analog thereof according to any one of claims 1-5, 8, and 9.

51. A method of therapeutically or prophylactically treating a patient which comprises the step of administering to said patient an effective amount of preptin or an analog thereof according to claim 6.

52. A method of therapeutically or prophylactically treating a patient which comprises the step of administering to said patient an effective amount of preptin or an analog thereof according to claim 7.

53. A method of therapeutically or prophylactically treating a patient which comprises the step of administering to said patient an effective amount of a salt according to claim 26.

54. A method of therapeutically or prophylactically treating a patient which comprises the step of administering to said patient an effective amount of a salt according to claim 27.

55. A method of therapeutically or prophylactically treating a patient which comprises the step of administering to said patient an effective amount of a salt according to claim 28.

56. A method of stimulating insulin secretion for a therapeutic or prophylactic purpose which comprises the step of administering to a patient in need of such therapy or prophylaxis an effective amount of preptin or an analog thereof according to any one of claims 1-5, 8, and 9.

57. A method of stimulating insulin secretion for a therapeutic or prophylactic purpose which comprises the step of administering to a patient in need of such therapy or prophylaxis an effective amount of preptin or an analog thereof according to claim 6.

58. A method of stimulating insulin secretion for a therapeutic or prophylactic purpose which comprises the step of administering to a patient in need of such therapy or prophylaxis an effective amount of preptin or an analog thereof according to claim 7.

59. A method of stimulating insulin secretion for a therapeutic or prophylactic purpose which comprises the step of administering to a patient in need of such therapy or prophylaxis an effective amount of a salt according to claim 26.

60. A method of stimulating insulin secretion for a therapeutic or prophylactic purpose which comprises the step of administering to a patient in need of such therapy or prophylaxis an effective amount of a. salt according to claim 27.

61. A method of stimulating insulin secretion for a therapeutic or prophylactic purpose which comprises the step of administering to a patient in need of such therapy or prophylaxis an effective amount of a salt according to claim 28.

62. A method of treating Type 2 diabetes mellitus which comprises the step of administering to a patient an effective amount of preptin or an analog thereof according to any one of claims 1-5, 8, and 9.

63. A method of treating Type 2 diabetes mellitus which comprises the step of administering to a patient an effective amount of preptin or an analog thereof according to claim 6.

64. A method of treating Type 2 diabetes mellitus which comprises the step of administering to a patient an effective amount of preptin or an analog thereof according to claim 7.

65. A method of treating Type 2 diabetes mellitus which comprises the step of administering to a patient an effective amount of a salt according to claim 26.

66. A method of treating Type 2 diabetes mellitus which comprises the step of administering to a patient an effective amount of a salt according to claim 27.

67. A method of treating Type 2 diabetes mellitus which comprises the step of administering to a patient an effective amount of a salt according to claim 28.

68. A method of treating a condition which results in or involves deficient insulin synthesis, secretion or action which comprises the step of administering to a patient an effective amount of preptin or an analog thereof according to any one of claims 1-5, 8, and 9.

69. A method of treating a condition which results in or involves deficient insulin synthesis, secretion or action which comprises the step of administering to a patient an effective amount of preptin or an analog thereof according to claim 6.

70. A method of treating a condition which results in or involves deficient insulin synthesis, secretion or action which comprises the step of administering to a patient an effective amount of preptin or an analog thereof according to claim 7.

71. A method of treating a condition which results in or involves deficient insulin synthesis, secretion or action which comprises the step of administering to a patient an effective amount of a salt according to claim 26.

72. A method of treating a condition which results in or involves deficient insulin synthesis, secretion or action which comprises the step of administering to a patient an effective amount of a salt according to claim 27.

73. A method of treating a condition which results in or involves deficient insulin synthesis, secretion or action which comprises the step of administering to a patient an effective amount of a salt according to claim 28.

74. Isolated antibodies which bind preptin or an analog thereof according to any one of claims 1-5, 8, and 9.

75. Isolated antibodies which bind preptin or an analog thereof according to claim 6.

76. Isolated antibodies which bind preptin or an analog thereof according to claim 7.

77. A monoclonal antibody which binds preptin or an analog thereof according to any one of claims 1-5, 8, and 9.

78. A monoclonal antibody which binds preptin or an analog thereof according to claim 6.

79. A monoclonal antibody which binds preptin or an analog thereof according to claim 7.

80. A polyclonal antibody which binds preptin or an analog thereof according to any one of claims 1-5, 8, and 9.

81. A polyclonal antibody which binds preptin or an analog thereof according to claim 6.

82. A polyclonal antibody which binds preptin or an analog thereof according to claim 7.

83. A monoclonal antibody which binds human preptin or an analog thereof according to any one of claims 3, 8 and 9.

84. A polyclonal antibody which binds human preptin or an analog thereof according to any one of claims 3, 8 and 9.

85. An immunological assay which employs an antibody according to claim 74.

86. An immunological assay which employs an antibody according to claim 75.

87. An immunological assay which employs an antibody according to claim 76.

88. An assay kit which includes an antibody according to claim 74.

89. An assay kit which includes an antibody according to claim 75.

90. An assay kit which includes an antibody according to claim 76.

91. A method of identifying a preptin agonist which comprises the steps of: testing the degree of insulin secretion induced by a predetermined concentration of preptin according to claim 1 in the presence and absence of a candidate agonist; and identifying as an agonist any compound which effects an increase in preptin-mediated insulin secretion.

92. A method of identifying a preptin antagonist which comprises the steps of testing the degree of insulin secretion induced by a predetermined concentration of preptin according to claim 1 in the presence and absence of a candidate antagonist; and identifying as an antagonist any compound which effects a decrease in preptin-mediated insulin secretion.

93. A method of modulating glucose mediated insulin secretion which comprises the step of administering to a patient an effective amount of a preptin agonist or a preptin antagonist.

94. An isolated polynucleotide coding for human preptin or a bioactive fragment thereof.

95. An isolated polynucleotide coding for human preptin analog or a bioactive fragment thereof.

96. An isolated polynucleotide coding for rat preptin or a bioactive fragment thereof.

97. An isolated polynucleotide coding for rat preptin analog or a bioactive fragment thereof.

98. An isolated polynucleotide coding for mouse preptin or a bioactive fragment thereof.

99. An isolated polynucleotide coding for mouse preptin analog or a bioactive fragment thereof.

100. An isolated polypeptide comprising an active fragment of preptin or an analog thereof.

101. The polypeptide according to claim 100 wherein said polypeptide is human, rat, or mouse.

102. A vector comprising a polynucleotide from any one of claims 94-99.

103. A host cell comprising a vector according to claim 102.

104. A host cell comprising a vector comprising a polynucleotide which codes for preptin or an analog thereof.

105. A method for generating monoclonal antibodies directed against preptin comprising:
administering preptin or a fragment thereof as an immunogen to mice;
obtaining splenocytes from said mice;
fusing said splenocytes with a myeloma cell line;
and culturing the fused splenocytes in a manner which promotes production of antibodies.


SEQUENCE LISTING (35 sequences)

SEQ ID NO: 1 - protein, 34 aa, Artificial Sequence
  Preptin
DVSTXXXVLPDXFPRYPVGKFFXXDTWXQSXXRL

SEQ ID NO: 2 - protein, 34 aa, Homo Sapien
  Preptin
DVSTPPTVLPDNFPRYPVGKFFQYDTWKQSTQRL

SEQ ID NO: 3 - protein, 34 aa, Rattus Sp.
  Preptin
DVSTSQAVLPDDFPRYPVGKFFKFDTWRQSAGRL

SEQ ID NO: 4 - protein, 34 aa, Mus Musculus
  Preptin
DVSTSQAVLPDDFPRYPVGKFFQYDTWRQSAGRL

SEQ ID NO: 5 - protein, 33 aa, Artificial Sequence
  Analog of human preptin
DVSTPPTVLPDNFPRYPVGKFFQYDTWKQSTQR

SEQ ID NO: 6 - protein, 32 aa, Artificial Sequence
  Analog of human preptin
DVSTPPTVLPDNFPRYPVGKFFQYDTWKQSTQ

SEQ ID NO: 7 - protein, 31 aa, Artificial Sequence
  Analog of human preptin
DVSTPPTVLPDNFPRYPVGKFFQYDTWKQST

SEQ ID NO: 8 - protein, 30 aa, Artificial Sequence
  Analog of human preptin
DVSTPPTVLPDNFPRYPVGKFFQYDTWKQS

SEQ ID NO: 9 - protein, 29 aa, Artificial Sequence
  Analog of human preptin
DVSTPPTVLPDNFPRYPVGKFFQYDTWKQ

SEQ ID NO: 10 - protein, 28 aa, Artificial Sequence
  Analog of human preptin
DVSTPPTVLPDNFPRYPVGKFFQYDTWK

SEQ ID NO: 11 - protein, 27 aa, Artificial Sequence
  Analog of human preptin
DVSTPPTVLPDNFPRYPVGKFFQYDTW

SEQ ID NO: 12 - protein, 26 aa, Artificial Sequence
  Analog of human preptin
DVSTPPTVLPDNFPRYPVGKFFQYDT

SEQ ID NO: 13 - protein, 25 aa, Artificial Sequence
  Analog of human preptin
DVSTPPTVLPDNFPRYPVGKFFQYD

SEQ ID NO: 14 - protein, 24 aa, Artificial Sequence
  Analog of human preptin
DVSTPPTVLPDNFPRYPVGKFFQY

SEQ ID NO: 15 - protein, 23 aa, Artificial Sequence
  Analog of human preptin
DVSTPPTVLPDNFPRYPVGKFFQ

SEQ ID NO: 16 - protein, 22 aa, Artificial Sequence
  Analog of human preptin
DVSTPPTVLPDNFPRYPVGKFF

SEQ ID NO: 17 - protein, 21 aa, Artificial Sequence
  Analog of human preptin
DVSTPPTVLPDNFPRYPVGKF

SEQ ID NO: 18 - protein, 20 aa, Artificial Sequence
  Analog of human preptin
DVSTPPTVLPDNFPRYPVGK

SEQ ID NO: 19 - protein, 19 aa, Artificial Sequence
  Analog of human preptin
DVSTPPTVLPDNFPRYPVG

SEQ ID NO: 20 - protein, 18 aa, Artificial Sequence
  Analog of human preptin
DVSTPPTVLPDNFPRYPV

SEQ ID NO: 21 - protein, 17 aa, Artificial Sequence
  Analog of human preptin
DVSTPPTVLPDNFPRYP

SEQ ID NO: 22 - protein, 16 aa, Artificial Sequence
  Analog of human preptin
DVSTPPTVLPDNFPRY

SEQ ID NO: 23 - protein, 15 aa, Artificial Sequence
  Analog of human preptin
DVSTPPTVLPDNFPR

SEQ ID NO: 24 - protein, 14 aa, Artificial Sequence
  Analog of human preptin
DVSTPPTVLPDNFP

SEQ ID NO: 25 - protein, 13 aa, Artificial Sequence
  Analog of human preptin
DVSTPPTVLPDNF

SEQ ID NO: 26 - protein, 12 aa, Artificial Sequence
  Analog of human preptin
DVSTPPTVLPDN

SEQ ID NO: 27 - protein, 11 aa, Artificial Sequence
  Analog of human preptin
DVSTPPTVLPD

SEQ ID NO: 28 - protein, 10 aa, Artificial Sequence
  Analog of human preptin
DVSTPPTVLP

SEQ ID NO: 29 - protein, 9 aa, Artificial Sequence
  Analog of human preptin
DVSTPPTVL

SEQ ID NO: 30 - protein, 8 aa, Artificial Sequence
  Analog of human preptin
DVSTPPTV

SEQ ID NO: 31 - protein, 7 aa, Artificial Sequence
  Analog of human preptin
DVSTPPT

SEQ ID NO: 32 - protein, 6 aa, Artificial Sequence
  Analog of human preptin
DVSTPP

SEQ ID NO: 33 - DNA, 102 bp, Homo Sapien
  Preptin
gacgtgtcgacccctccgaccgtgcttccggacaacttccccagataccccgtgggcaagttcttccaatatgacacctggaagcagtccacccagcgcctg

SEQ ID NO: 34 - DNA, 102 bp, Rattus Sp.
  Preptin
gacgtgtctacctctcaggccgtacttccggacgacttccccagataccccgtgggcaagttcttcaaattcgacacctggagacagtccgcgggacgcctg

SEQ ID NO: 35 - DNA, 102 bp, Mus Musculus
  Preptin
gacgtgtctacctctcaggccgtacttccggacgacttccccagataccccgtgggcaagttcttccaatatgacacctggagacagtccgcgggacgcctg